(19) United States
(12) Patent Application Publication
(10) Pub. No.: US 2025/0073147 A1
(43) Pub. Date: Mar. 6, 2025

(54) PROCESS FOR COLOURING THE HAIR COMPRISING THE APPLICATION OF A COMPOSITION COMPRISING A (POLY)CARBODIIMIDE COMPOUND, AND THE APPLICATION OF A COMPOSITION COMPRISING A SILICONE ELASTOMER CONTAINING CARBOXYLIC ACID FUNCTIONS

(71) Applicant: L'Oreal, Paris (FR)

(72) Inventors: Alexis Liard, St Ouen (FR); Charlotte Delostal, St Ouen (FR)

(73) Assignee: L'Oreal, Paris (FR)

(21) Appl. No.: 18/719,177

(22) PCT Filed: Dec. 12, 2022
(86) PCT No.: PCT/EP2022/085399

§ 371 Date: Jun. 12, 2024

(30) Foreign Application Priority Data
Dec. 13, 2021 (FR) 2113386

(51) Int. Cl. A61K 8/44; A61K 8/89; A61Q 5/06; A61Q 5/10

(52) U.S. Cl. CPC A61K 8/44; A61K 8/89; A61Q 5/065; A61Q 5/10; A61K 2800/4324; A61K 2800/592; A61K 2800/87; A61K 2800/882

(57) ABSTRACT

The present invention relates to a process for colouring the hair comprising the following steps: a) the application to the hair of at least one composition C comprising: —one or more (poly)carbodiimide compounds; and b) the application to the hair of at least one composition D comprising at least one silicone elastomer containing carboxylic acid functions; composition C and/or composition D comprising at least one colouring agent chosen from pigments, direct dyes and mixtures thereof.

The present invention relates to a process for colouring the hair comprising the application to the hair of at least one composition C comprising one or more (poly)carbodiimide compounds; and the application to the hair of at least one composition D comprising at least one silicone elastomer containing carboxylic acid functions. Composition C and/or composition D comprise at least one colouring agent chosen from pigments, direct dyes and mixtures thereof.

### TECHNICAL FIELD

In the field of colouring keratinous hair fibres, in particular human keratinous hair fibres, it is already known practice to colour keratinous hair fibres via various techniques using direct dyes or pigments for non-permanent colouring, or dye precursors for permanent colouring.

There are essentially three types of process for colouring the hair:
a) “permanent” colouring, the function of which is to afford a substantial modification to the natural colour and which uses oxidation dyes which penetrate into the hair fibre and forms the dye via an oxidative condensation process;
b) non-permanent, semi-permanent or direct colouring, which does not use the oxidative condensation process and withstands four or five shampoo washes; it consists in colouring keratinous fibres with colour compositions containing direct dyes;
c) temporary colouring, which gives rise to a modification of the natural colour of the hair that remains from one shampoo wash to the next, and which serves to enhance or correct a shade that has already been obtained. It may also be likened to a “makeup” process.

For this last type of colouring, it is known practice to use pigments. Specifically, the use of pigment on the surface of keratinous fibres generally makes it possible to obtain colourings that are visible on dark hair, since the surface pigment masks the natural colour of the fibre. However, the colourings obtained via this colouring method have the drawback of having poor resistance to shampoo washing and also to external agents such as sebum, perspiration, brushing and/or rubbing.

There is thus still a need for a process for colouring the hair, which has the advantage of obtaining a uniform-coloured coating on the hair, while at the same time forming a coating that is persistent with respect to shampoo washing and to the various attacking factors to which the hair may be subjected such as brushing and/or friction without degradation of the cosmetic properties of the hair.

Thus, the aim of the present invention is to develop a process for colouring the hair, which has the advantage of obtaining a smooth and uniform coloured coating on the hair, while at the same time forming a coating that is persistent with respect to shampoo washing and to the various attacking factors to which the hair may be subjected such as brushing and/or friction without degradation of the cosmetic properties of the hair.

### DISCLOSURE OF THE INVENTION

One subject of the present invention is thus a process for colouring the hair comprising the following steps:
a) the application to the hair of at least one composition C comprising:
   one or more (poly)carbodiimide compounds; and
b) the application to the hair of at least one composition D comprising at least one silicone elastomer containing carboxylic acid functions;
composition C and/or composition D comprising at least one colouring agent chosen from pigments, direct dyes and mixtures thereof.

By means of the process for colouring the hair according to the invention, coloured coatings are obtained on the hair that make it possible to obtain a colouring that is visible on all types of hair in a manner that is persistent with respect to shampoo washing, while at the same time preserving the physical qualities of the hair.

Such a coating may be resistant to the external attacking factors to which the hair may be subjected, such as blow-drying and perspiration. It makes it possible in particular to obtain a uniform deposit.

For the purposes of the present invention, the term “colouring that is persistent with respect to shampoo washing” means that the colouring obtained persists after one shampoo wash, preferably after three shampoo washes, more preferentially after five shampoo washes.

The term “keratinous hair fibres” means the hair. In other words, the expressions “keratinous hair fibres” and “hair” are equivalent in the remainder of the description.

For the purposes of the present invention, the term “hair” means the hair on the head. This term does not correspond to body hair, the eyebrows or the eyelashes.

The term “at least one” means one or more.

Unless indicated otherwise, the limits of a range of values are included in this range, in particular in the expressions “between” and “ranging from . . . to . . . ”.

The invention is not limited to the illustrated examples. The characteristics of the various examples may notably be combined within variants which are not illustrated.

For the purposes of the present invention and unless otherwise indicated:
an “alkyl” radical denotes a linear or branched saturated radical containing, for example, from 1 to 20 carbon atoms;
an “aminoalkyl” radical denotes an alkyl radical as defined previously, said alkyl radical comprising an NH₂ group;
a “hydroxyalkyl” radical denotes an alkyl radical as defined previously, said alkyl radical comprising an OH group;
an “alkylene” radical denotes a linear or branched divalent saturated C₂-C₄ hydrocarbon-based group such as methylene, ethylene or propylene;
a “cycloalkyl” or “alicycloalkyl” radical denotes a cyclic saturated monocyclic or bicyclic, preferably monocyclic, hydrocarbon-based group comprising from 1 to 3 rings, preferably 2 rings, and comprising from 3 to 24 carbon atoms, in particular comprising from 3 to 20 carbon atoms, more particularly from 3 to 13 carbon atoms, even more particularly from 3 to 12 carbon atoms, preferably between 5 and 10 carbon atoms, such as cyclopropyl, cyclopentyl, cyclohexyl, cycloheptyl or norbornyl, in particular cyclopropyl, cyclopentyl or cyclohexyl, it being understood that the cycloalkyl radical may be substituted with one or more (C₁-C₄)alkyl groups such as methyl; preferably, the cycloalkyl radical is then an isobornyl group;
a “cycloalkylene” radical denotes a divalent cycloalkyl group with “cycloalkyl” as defined previously, preferably of C₃-C₁₂;
an “aryl” radical is a monocyclic, bicyclic or tricyclic, fused or non-fused, unsaturated and aromatic hydrocarbon-based cyclic radical, comprising from 6 to 14 carbon atoms, preferably between 6 and 12 carbon atoms; preferably, the aryl group comprises 1 ring of 6 carbon atoms such as phenyl, naphthyl, anthryl, phenanthryl and biphenyl, it being understood that the aryl radical may be substituted with one or more (C₁-C₄)alkyl groups such as methyl, preferably tolyl, xylyl, or methylnaphthyl; preferably, the aryl group represents phenyl;
an “arylene” radical is a divalent aryl radical with “aryl” as defined previously; preferably, arylene represents phenylene;
a “heterocyclic” radical denotes a saturated or unsaturated, non-aromatic or aromatic, monocyclic or polycyclic hydrocarbon-based radical, comprising one or more heteroatoms, preferably from 1 to 5 atoms chosen from O, S or N, including from 3 to 20 ring members, preferably between 5 and 10 ring members, such as imidazolyl, pyrrolyl and furanyl;
a “heterocycloalkylene” radical is a divalent heterocyclic group with “heterocyclic” as defined previously;
an “aryloxy” radical denotes an aryl-oxy with “aryl” as defined previously;
an “alkoxy” radical denotes an alkyl-oxy radical with “alkyl” as defined previously;
an “acyloxy” radical denotes an ester radical R—C(O)—O— with R being an alkyl group as defined previously;
a “reactive” group is a group that is capable of forming a covalent bond with another identical or different group, by chemical reaction.

Unless indicated otherwise, when compounds are mentioned in the present application, this is understood to also mean the optical isomers thereof, the geometric isomers thereof, the tautomers thereof and the salts thereof, alone or in a mixture.

### DETAILED DESCRIPTION OF THE INVENTION

### Process According to the Invention:

### Composition C:

### Polycarbodiimide Compound

Composition C employed within the context of the process according to the invention comprises at least one (poly)carbodiimide compound.

The composition may comprise at least two different (poly)carbodiimide compounds, present as a mixture in the composition.

The term “(poly)carbodiimide compound” means a compound comprising one or more carbodiimide groups, preferably at least two carbodiimide groups, more preferentially at least three carbodiimide groups; in particular, the number of carbodiimide groups does not exceed 200, preferably 150, more preferentially 100.

The term “carbodiimide group” means a divalent linear triatomic fraction of general formula —(N═C═N)—.

The (poly)carbodiimide compound(s) according to the invention may optionally comprise in their structure one or more reactive groups different from carbodiimide groups, chosen from alkoxysilyl, hydroxysilyl, acetoxysilyl, vinylsilyl, acrylalkylsilyl, methacrylalkylsilyl, crotonylalkylsilyl, carboxyanhydridoalkylsilyl, carboxyalkylsilyl, hydroxyalkylsilyl, aldehydoalkylsilyl, mercaptoalkylsilyl, norbornenylsilyl, acylpentadienylalkylsilyl, maleimidoalkylsilyl, sulfonylalkylsilyl, (meth)acrylalkyl, crotonylalkyl, alkylepoxide such as propylepoxide or butylepoxide and azacyclopropane groups.

The reactive group(s) other than the carbodiimide groups may be side or end groups. Preferably, the (poly)carbodiimide compound(s) comprise one or more end groups different from carbodiimide groups, preferably one or more end groups chosen from alkoxysilyl, hydroxysilyl, acetoxysilyl, vinylsilyl, acrylalkylsilyl, methacrylalkylsilyl, crotonylalkylsilyl, carboxyanhydridoalkylsilyl, carboxyalkylsilyl, hydroxyalkylsilyl, aldehydoalkylsilyl, mercaptoalkylsilyl, norbornenylsilyl, acylpentadienylalkylsilyl, maleimidoalkylsilyl, sulfonylalkylsilyl, (meth)acrylalkyl, crotonylalkyl, alkylepoxide such as propylepoxide or butylepoxide and azacyclopropane groups.

According to a particular embodiment, the (poly)carbodiimide compound is chosen from the compounds of formula (I) below:

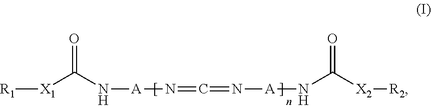

in which:
X₁ and X₂ independently represent an oxygen atom O, a sulfur atom S or an NH group;
R₁ and R₂ independently represent a group chosen from a hydrocarbon-based radical, preferably alkyl, optionally interrupted with one or more heteroatoms, a group chosen from alkoxysilyl, hydroxysilyl, acetoxysilyl, vinylsilyl, acrylalkylsilyl, methacrylalkylsilyl, crotonylalkylsilyl, carboxyanhydridoalkylsilyl, carboxyalkylsilyl, hydroxyalkylsilyl, aldehydoalkylsilyl, mercaptoalkylsilyl, norbornenylsilyl, acylpentadienylalkylsilyl, maleimidoalkylsilyl, sulfonylalkylsilyl, (meth)acrylalkyl, crotonylalkyl, alkylepoxide such as propylepoxide or butylepoxide and azacyclopropane groups, and a hydrocarbon-based radical, preferably alkyl, optionally interrupted with one or more heteroatoms and with one or more groups chosen from alkoxysilyl, hydroxysilyl, acetoxysilyl, vinylsilyl, acrylalkylsilyl, methacrylalkylsilyl, crotonylalkylsilyl, carboxyanhydridoalkylsilyl, carboxyalkylsilyl, hydroxyalkylsilyl, aldehydoalkylsilyl, mercaptoalkylsilyl, norbornenylsilyl, acylpentadienylalkylsilyl, maleimidoalkylsilyl, sulfonylalkylsilyl, (meth)acrylalkyl, crotonylalkyl, alkylepoxide such as propylepoxide or butylepoxide and azacyclopropane groups;
n denotes an integer ranging from 1 to 1000; and
A is a monomer chosen from the compounds below:

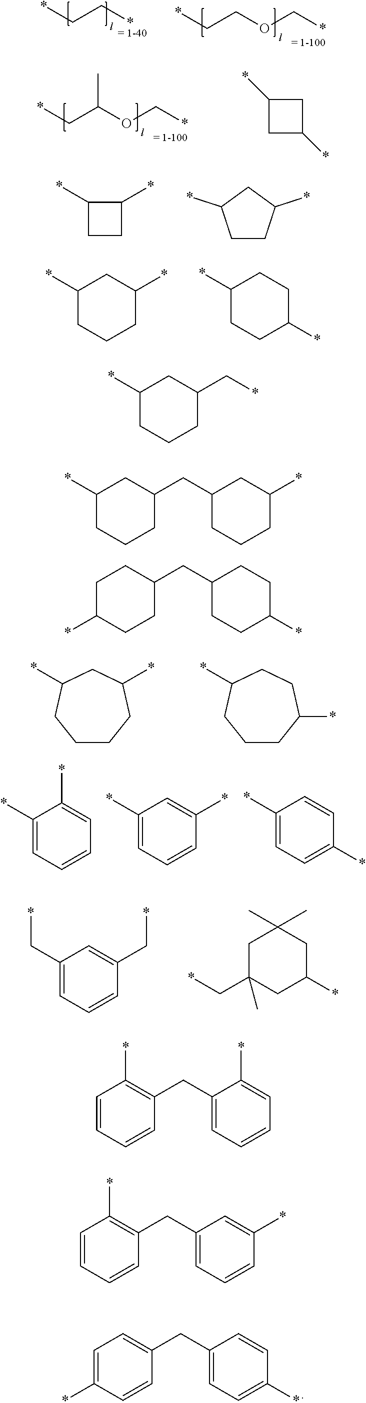

According to another embodiment, the (poly)carbodiimide compound is chosen from the compounds of formula (I′) below:

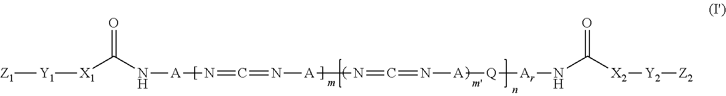

in which:
X₁ and X₂ independently represent an oxygen atom O, a sulfur atom S or an NH group;
Y₁ and Y₂ independently represent a divalent organic radical chosen from a saturated C₁ to C₃₆ aliphatic group or a C₆ to C₂₄ aromatic or alkylaromatic group, the aliphatic or aromatic group optionally comprising one or more non-pendent heteroatoms, such as a nitrogen atom, an oxygen atom, a sulfur atom, or combinations thereof,
Z₁ and Z₂ independently represent a reactive end group or an inert end group;
as inert end group, Z₁ and Z₂ may represent, independently, a saturated, linear or branched or cyclic C₁ to C₅₀ aliphatic group, or a C₆ to Cis aromatic group, said aliphatic and aromatic groups optionally comprising from 1 to 10 heteroatoms chosen from nitrogen, oxygen, sulfur and combinations thereof, and the aliphatic or aromatic group may be partially or totally fluorinated; in this variant, Z₁ and Z₂ comprise a bonding group CG connecting Z₁ to Y₁ and Z₂ to Y₂, the group CG possibly being a single covalent bond, a saturated C—C bond, an unsaturated covalent C—C bond, an amide group, an ester group, a carbonate group, a thioester group, an ether group, a urethane group, a thiourethane group or a urea group;
as reactive end group, Z₁ and Z₂ may be chosen from alkoxysilyl, hydroxysilyl, acetoxysilyl, vinylsilyl, acrylalkylsilyl, methacrylalkylsilyl, crotonylalkylsilyl, carboxyanhydridoalkylsilyl, carboxyalkylsilyl, hydroxyalkylsilyl, aldehydoalkylsilyl, mercaptoalkylsilyl, norbornenylsilyl, acylpentadienylalkylsilyl, maleimidoalkylsilyl, sulfonylalkylsilyl, (meth)acrylalkyl, crotonylalkyl, alkylepoxide such as propylepoxide or butylepoxide and azacyclopropane groups;
Q represents an organopolymer or an organooligomer comprising repeating units of saturated, linear or branched or cyclic aliphatic groups, or of aromatic groups or alkylaromatic groups, coupled via carbonate, ester, ether, amide, urethane or urea repeating bonds or combinations thereof,
A represents a divalent aliphatic, aromatic, alkylaromatic or linear, saturated, branched or cyclic radical containing from 2 to 30 carbon atoms, which may optionally comprise one or more non-pendent heteroatoms such as a nitrogen atom, an oxygen atom, a sulfur atom, or combinations thereof, in the aliphatic chain or the aromatic chain;
r denotes an integer equal to 0 or 1;
m denotes an integer ranging from 0 to 1000, preferably equal to 0 or 1;
m′ denotes an integer ranging from 0 to 1000, preferably equal to 0 or 1;
n denotes an integer ranging from 0 to 1000, preferably equal to 0 or 1, with m+(m′*n)≥2.

Preferably, Z₁ and Z₂ independently represent a reactive end group; more preferentially, Z₁ and Z₂ independently represent a group chosen from alkoxysilyl, hydroxysilyl, acetoxysilyl, vinylsilyl, acrylalkylsilyl, methacrylalkylsilyl, crotonylalkylsilyl, carboxyanhydridoalkylsilyl, carboxyalkylsilyl, hydroxyalkylsilyl, aldehydoalkylsilyl, mercaptoalkylsilyl, norbornenylsilyl, acylpentadienylalkylsilyl, maleimidoalkylsilyl, sulfonylalkylsilyl, (meth)acrylalkyl, crotonylalkyl, alkylepoxide such as propylepoxide or butylepoxide and azacyclopropane groups.

Such (poly)carbodiimide compounds are sold, for example, by the company Stahl B.V, under the name Permutex XR, or under the name RelcaLink10. or under the name Picassian XL and by the company Nisshinbo under the name Carbodilite with the series V-02, V-02-L2, SV-02, E-02, V-10, SW-12G, E-03A, E-04DG-T, E-05, V-04, V-02B, V-04PF, V-05.

Preferably, the (poly)carbodiimide compound(s) are chosen from the compounds of formula (II) below:

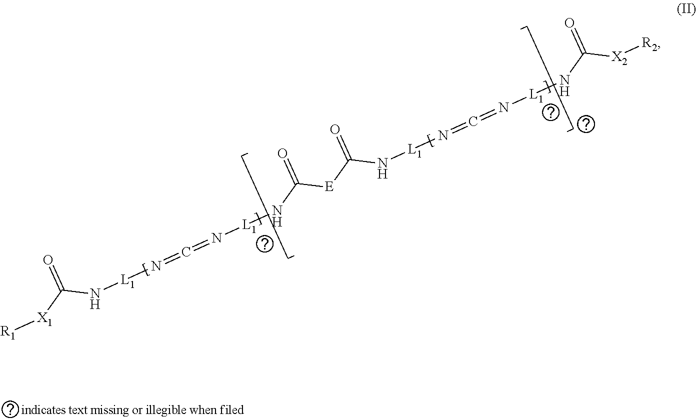

in which:
X₁ and X₂ independently represent an oxygen atom O, a sulfur atom S or an NH group;
R₁ and R₂ independently represent a hydrocarbon-based radical optionally interrupted with one or more heteroatoms;
n and z denote an integer ranging from 1 to 20, with n+z≥2 and w denotes an integer ranging from 1 to 3;
L₁ independently represents a C₁-C₁₅ divalent aliphatic hydrocarbon-based radical, a C₃-C₁₅ cycloalkylene radical, a C₃-C₁₂ heterocycloalkylene group or a C₆-C₁₄ arylene group, and mixtures thereof;
E independently represents a group chosen from:
—O—R₃—O—; —S—R₄—S—; —R₅—N(R₆)—R₄—N(R₆)—R₅—;
   in which R₃ and R₄ independently represent a divalent hydrocarbon-based radical optionally interrupted with one or more heteroatoms;
R₅ independently represents a covalent bond or a saturated divalent hydrocarbon-based radical, optionally interrupted with one or more heteroatoms;
R₆ independently represents a hydrogen atom or a hydrocarbon-based radical, optionally interrupted with one or more heteroatoms.

The term “hydrocarbon-based radical” means a saturated or unsaturated, linear or branched radical containing from 1 to 300 carbon atoms, preferably from 1 to 250 carbon atoms, more preferentially from 1 to 200 carbon atoms. Preferably, the hydrocarbon-based radical is a saturated linear radical.

The hydrocarbon-based radical may comprise one or more cyclic groups.

The hydrocarbon-based radical may be interrupted with one or more heteroatoms, in particular chosen from O, S or N and/or substituted with one or more cations, anions or zwitterions or cationic groups such as ammonium, anionic groups such as carboxylate, or zwitterionic groups, and/or comprising a metal ion which may be incorporated in the form of a salt.

The term “heteroatom(s)” means an oxygen 0, sulfur S or nitrogen N atom, and also halogen atoms such as Cl, F, Br and I. If the heteroatom is included in the chain of the hydrocarbon-based radical, the heteroatom is preferably chosen from oxygen O, sulfur S or nitrogen N atoms.

Preferably, X₁ and X₂ independently represent an oxygen atom.

Preferably, R₁ and R₂ are independently chosen from dialkylamino alcohols, alkyl esters of hydroxycarboxylic acid and monoalkyl ethers of (poly)alkylene glycol, in which a hydroxyl group has been removed, and mixtures thereof.

In a preferred embodiment, R₁ and R₂ are independently chosen from groups (i) to (iv) below:
(i) the compound of formula (III) below:

R₇—O—C(O)—C(R₈)(H)— (III),

in which R₇ represents a C₁-C₃ alkyl group and R₈ represents a hydrogen atom or a C₁-C₃ alkyl group; preferably, R₇ is a methyl and R₈ is a hydrogen atom or a methyl.
(ii) the compound of formula (IV) below:

R₉—[O—CH₂—C(H)(R₁₀)]ₚ— (IV),

in which R₉ represents a C₁-C₄ alkyl group, R₁₀ represents a hydrogen atom or a C₁-C₄ alkyl group and p denotes an integer ranging from 1 to 3; preferably, R₉ is a methyl, ethyl or butyl, R₁₀ is a hydrogen atom or a methyl and p is equal to 1.
(iii) the compound of formula (V) below:

(R₁₁)₂N—CH₂—C(H)(R₁₂)— (V),

in which R₁₁ represents a C₁-C₄ alkyl group and R₁₂ represents a hydrogen atom or a C₁-C₄ alkyl group; preferably, R₁₁ is a methyl, ethyl or butyl and R₁₂ is a hydrogen atom or a methyl.
(iv) the compound of formula (VI) below:

R₁₃—[O—CH₂—C(H)(R₁₄)]_{q}— (VI),

in which R₁₃ represents a C₁-C₄ alkyl group or a phenyl, R₁₄ represents a hydrogen atom or a C₁-C₄ alkyl group and q denotes an integer ranging from 4 to 30; preferably, R₁₃ is a methyl, ethyl or butyl and R₁₄ is a hydrogen atom or a methyl.

Preferably, R₁ and R₂ independently represent a compound of formula (VI) in which R₁₃ represents a C₁-C₄ alkyl group or a phenyl, preferably a C₁-C₄ alkyl group, more preferentially a methyl, R₁₄ represents a hydrogen atom or a C₁-C₄ alkyl group, preferably a hydrogen atom and q denotes an integer ranging from 4 to 30.

According to an alternative embodiment, R₁ and R₂ are different and one of the radicals R₁ or R₂ represents a compound of formula (IV) as described above and the other radical R₁ or R₂ represents a compound of formula (VI) as described above.

Preferably, in formula (IV), R₉ is a methyl, ethyl or butyl and R₁₀ is a hydrogen atom or a methyl and p is equal to 1.

Preferably, in formula (VI), R₁₃ is a methyl, ethyl or butyl and R₁₄ is a hydrogen atom or a methyl and q denotes an integer ranging from 4 to 30.

According to another alternative embodiment, R₁ and R₂ are identical and represent a compound of formula (VI) in which R₁₃ represents a C₁-C₄ alkyl group or a phenyl, preferably a C₁-C₄ alkyl group, more preferentially a methyl, R₁₄ represents a hydrogen atom or a C₁-C₄ alkyl group, preferably a hydrogen atom and q denotes an integer ranging from 4 to 30.

Preferably, n denotes an integer ranging from 1 to 20, more preferentially from 2 to 20.

Preferably, z denotes an integer ranging from 1 to 20, more preferentially from 2 to 20.

Preferably, w is equal to 1.

Preferably, w is equal to 1, n+z denotes an integer ranging from 4 to 10.

Preferably, L₁ is chosen from a C₁-C₁₅ divalent aliphatic hydrocarbon-based radical such as methylene, ethylene and propylene, a C₃-C₁₅ cycloalkylene radical such as cyclopentylene, cycloheptylene and cyclohexylene, a C₃-C₁₂ heterocycloalkylene group such as imidazolene, pyrrolene and furanylene, or a C₆-C₁₄ arylene group such as phenylene, and mixtures thereof.

For example, L₁ may be chosen from a radical derived from tolylene diisocyanate, hexamethylene diisocyanate, xylylene diisocyanate, 2,2,4-trimethylhexamethylene diisocyanate, 1,12-dodecane diisocyanate, norbornane diisocyanate, 2,4-bis(8-isocyanatooctyl)-1,3-dioctylcyclobutane, 4,4′-dicyclohexylmethane diisocyanate, tetramethylxylylene diisocyanate, isophorone diisocyanate, 1,5-napththylene diisocyanate, 4,4′-diphenylmethane diisocyanate, 4,4′-diphenyldimethylmethane diisocyanate and phenylene diisocyanate, and mixtures thereof.

Preferably, L₁ is chosen from a C₃-C₁₅ cycloalkylene radical or a C₆-C₁₄ arylene group, and mixtures thereof, such as the compounds of formula (VII) below:

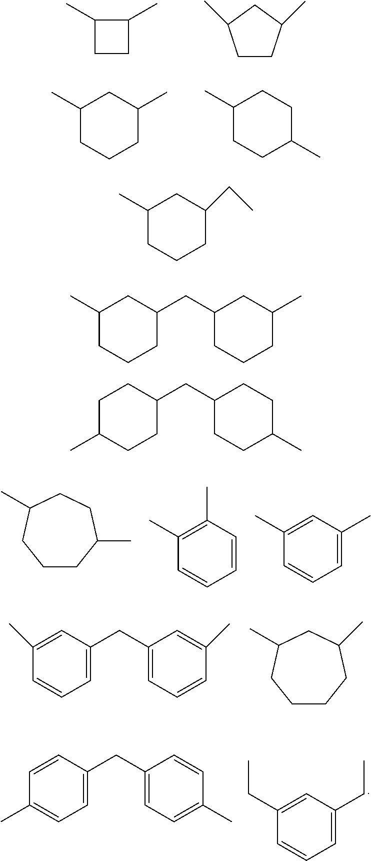

Preferably, L1 is 4,4-dicyclohexylenemethane corresponding to formula (VIII) below:

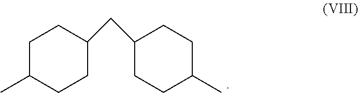

According to another embodiment, when L1 is a C₆-C₁₄ arylene group, L₁ is not the m-tetramethylxylylene radical represented by formula (IX) below:

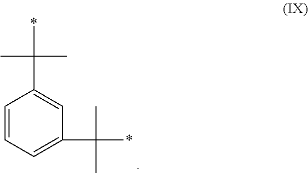 [SMILES: *C(C)(C)c1cccc(C(*)(C)C)c1]

As indicated previously, E independently represents a group chosen from:
—O—R₃—O—; —S—R₄—S—; —R₅—N(R₆)—R₄—N(R₆)—R₅—;
   in which R₃ and R₄ independently represent a divalent hydrocarbon-based radical optionally interrupted with one or more heteroatoms;
R₅ independently represents a covalent bond or a saturated divalent hydrocarbon-based radical, optionally interrupted with one or more heteroatoms; and
R₆ independently represents a hydrogen atom or a hydrocarbon-based radical, optionally interrupted with one or more heteroatoms.

Preferably, R₃ and R₄ are independently chosen from a C₆-C₁₄ arylene radical such as phenylene, a C₃-C₁₂ cycloalkylene radical such as cyclopropylene and cyclobutylene, a linear or branched C₁-C₁₈ alkylene radical such as methylene and ethylene, optionally interrupted with one or more heteroatoms, and mixtures thereof.

More preferentially, R₃ and R₄ are independently chosen from a linear or branched C₁-C₁₈ alkylene radical such as methylene, butylene, propylene or ethylene, optionally interrupted with one or more heteroatoms.

Preferably, when R₅ is not a covalent bond, R₅ is chosen from a C₆-C₁₄ arylene radical such as phenylene, a C₃-C₁₂ cycloalkylene radical such as cyclopropylene and cyclobutylene, a linear or branched C₁-C₁₈ alkylene radical such as methylene and ethylene, optionally interrupted with one or more heteroatoms, and mixtures thereof.

Preferably, R₆ is chosen from a C₆-C₁₄ arylene radical such as phenylene, a C₃-C₁₂ cycloalkylene radical such as cyclopropylene and cyclobutylene, a linear or branched C₁-C₁₈ alkylene radical such as methylene and ethylene, optionally interrupted with one or more heteroatoms, and mixtures thereof.

Preferably, E represents a group —O—R₃—O— in which R₃ is chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof.

More preferentially, E represents a group —O—R₃—O— in which R₃ represents a linear or branched C₁-C₁₈ alkylene radical such as methylene, butylene, propylene or ethylene, optionally interrupted with one or more heteroatoms.

According to a particular embodiment, the (poly)carbodiimide compound is a copolymer derived from α-methylstyryl isocyanates of formula (X) below:

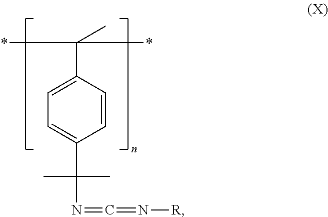

in which R independently represents an alkyl group containing from 1 to 24 carbon atoms, a cycloalkyl group containing from 3 to 24 carbon atoms or an aryl group containing from 6 to 24 carbon atoms, and n denotes an integer ranging from 2 to 100.

In this embodiment, the term “alkyl group” is as defined previously.

In this embodiment, the term “cycloalkyl group” is as defined previously.

In this embodiment, n may denote an integer ranging from 2 to 50, preferably from 3 to 30 and even more preferentially from 5 to 10.

According to another particular embodiment, the (poly)carbodiimide compound is a compound of formula (XI) below:

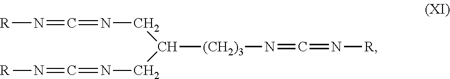

in which R independently represents an alkyl group containing from 1 to 24 carbon atoms, a cycloalkyl group containing from 3 to 24 carbon atoms or an aryl group containing from 6 to 24 carbon atoms.

The “alkyl group”, the “cycloalkyl group” and the “aryl group” are as defined previously.

According to a preferred embodiment, the (poly)carbodiimide compound is chosen from the compounds of formula (I) or of formula (II) in which:
X₁ and X₂ independently represent an oxygen atom;
R₁ and R₂ are independently chosen from dialkylamino alcohols, alkyl esters of hydroxycarboxylic acid and monoalkyl ethers of (poly)alkylene glycol, in which a hydroxyl group has been removed, and mixtures thereof, preferably monoalkyl ethers of (poly)alkylene glycol, in which a hydroxyl group has been removed, more preferentially the compound of formula (VI) as described previously in which R₁₃ represents a C₁-C₄ alkyl group or a phenyl, preferably a C₁-C₄ alkyl group, more preferentially a methyl, R₁₄ represents a hydrogen atom or a C₁-C₄ alkyl group, preferably a hydrogen atom, and q denotes an integer ranging from 4 to 30;
n and z, when they are present, denote an integer ranging from 1 to 20, with n+z≥2 and w is equal to 1;
L₁, when it is present, is chosen from a C₁-C₁₅ divalent aliphatic hydrocarbon-based radical, a C₃-C₁₅ cycloalkylene radical, a C₃-C₁₂ heterocycloalkylene group or a C₆-C₁₄ arylene group, and mixtures thereof, preferably a C₃-C₁₅ cycloalkylene radical;
A, when it is present, is chosen from a C₁-C₁₅ divalent aliphatic hydrocarbon-based radical, a C₃-C₁₅ cycloalkylene radical, a C₃-C₁₂ heterocycloalkylene group or a C₆-C₁₄ arylene group, and mixtures thereof, preferably a C₃-C₁₅ cycloalkylene radical;
E, when it is present, independently represents a group chosen from:
—O—R₃—O—; —S—R₄—S—; —R₅—N(R₆)—R₄—N(R₆)—R₅—;
   in which R₃ and R₄ are independently chosen from a C₆-C₁₄ arylene radical, a C₃-C₁₂ cycloalkylene radical, a linear or branched C₁-C₁₅ alkylene radical, optionally interrupted with one or more heteroatoms, and mixtures thereof;
when R₅ is not a covalent bond, R₅, when it is present, is chosen from a C₆-C₁₄ arylene radical, a C₃-C₁₂ cycloalkylene radical, a linear or branched C₁-C₁₅ alkylene radical, optionally interrupted with one or more heteroatoms, and mixtures thereof, and
R₆, when it is present, is chosen from a C₆-C₁₄ arylene radical, a C₃-C₁₂ cycloalkylene radical, a linear or branched C₁-C₁₅ alkylene radical, optionally interrupted with one or more heteroatoms, and mixtures thereof.

Preferably, the (poly)carbodiimide compound is chosen from the compounds of formula (II) in which:
X₁ and X₂ independently represent an oxygen atom;
R₁ and R₂ are independently chosen from dialkylamino alcohols, alkyl esters of hydroxycarboxylic acid and monoalkyl ethers of (poly)alkylene glycol, in which a hydroxyl group has been removed, and mixtures thereof,
n and z denote an integer ranging from 1 to 20, with n+z≥2 and w is equal to 1;
L₁ is chosen from a C₁-C₁₈ divalent aliphatic hydrocarbon-based radical, a C₃-C₁₅ cycloalkylene radical, a C₃-C₁₂ heterocycloalkylene group or a C₆-C₁₄ arylene group, and mixtures thereof;
E independently represents a group chosen from:
—O—R₃—O—; —S—R₄—S—; —R₅—N(R₆)—R₄—N(R₆)—R₅—;
   in which R₃ and R₄ are independently chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof;
when R₅ is not a covalent bond, R₅ is chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof, and
R₆ is chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof.

More preferentially, the (poly)carbodiimide compound is chosen from the compounds of formula (II) in which:
X₁ and X₂ independently represent an oxygen atom;
R₁ and R₂ are, independently, monoalkyl ethers of (poly)alkylene glycol, in which a hydroxyl group has been removed;
n and z denote an integer ranging from 1 to 20, with n+z≥2 and w is equal to 1;
L₁ is a C₃-C₁₅ cycloalkylene radical;
E independently represents a group chosen from:
—O—R₃—O—; —S—R₄—S—; —R₅—N(R₆)—R₄—N(R₆)—R₅—;
   in which R₃ and R₄ are independently chosen from a C₆-C₁₄ arylene radical, a C₃-C₁₂ cycloalkylene radical, a linear or branched C₁-C₁₅ alkylene radical, optionally interrupted with one or more heteroatoms, and mixtures thereof;
when R₅ is not a covalent bond, R₅ is chosen from a C₆-C₁₄ arylene radical, a C₃-C₁₂ cycloalkylene radical, a linear or branched C₁-C₁₅ alkylene radical, optionally interrupted with one or more heteroatoms, and mixtures thereof; and
R₆ is chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof.

Even more preferentially, the (poly)carbodiimide compound is chosen from the compounds of formula (II) in which:
X₁ and X₂ independently represent an oxygen atom;
R₁ and R₂ independently represent the compound of formula (VI) below:

R₁₃—[O—CH₂—C(H)(R₁₄)]_{q}— (VI),
in which R₁₃ represents a C₁-C₄ alkyl group or a phenyl, preferably a C₁-C₄ alkyl group, more preferentially a methyl, R₁₄ represents a hydrogen atom or a C₁-C₄ alkyl group, preferably a hydrogen atom and q denotes an integer ranging from 4 to 30;
n and z denote an integer ranging from 2 to 20, with n+z ranging from 4 to 10 and w is equal to 1;
L₁ is a C₃-C₁₅ cycloalkylene radical such as cyclopentylene, cycloheptylene, cyclohexylene and 4,4-dicyclohexylenemethane; and
E represents a group —O—R₃—O— in which R₃ is chosen from a C₆-C₁₄ arylene radical, a C₃-C₁₂ cycloalkylene radical, a linear or branched C₁-C₁₅ alkylene radical, optionally interrupted with one or more heteroatoms, and mixtures thereof.

Even more preferentially, the (poly)carbodiimide compound is chosen from the compounds of formula (II) in which:
X₁ and X₂ independently represent an oxygen atom;
R₁ and R₂ independently represent the compound of formula (VI) below:

R₁₃—[O—CH₂—C(H)(R₁₄)]_{q}— (VI)

in which R₁₃ represents a C₁-C₄ alkyl group or a phenyl, preferably a C₁-C₄ alkyl group, more preferentially a methyl, R₁₄ represents a hydrogen atom or a C₁-C₄ alkyl group, preferably a hydrogen atom and q denotes an integer ranging from 4 to 30;
n and z denote an integer ranging from 2 to 20, with n+z ranging from 4 to 10 and w is equal to 1;
L₁ is a C₃-C₁₅ cycloalkylene radical such as cyclopentylene, cycloheptylene, cyclohexylene and 4,4-dicyclohexylenemethane, preferably 4,4-dicyclo-hexylene-methane; and
E represents a group —O—R₃—O— in which R₃ represents a linear or branched C₁-C₁₈ alkylene radical such as methylene, propylene, butylene or ethylene, optionally interrupted with one or more heteroatoms.

According to a preferred embodiment, the (poly)carbodiimide compound is a compound of formula (XII) below:

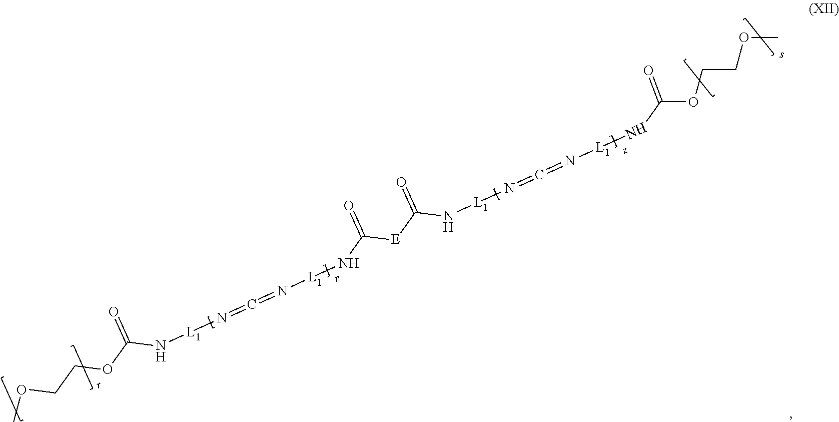

in which L1 is 4,4-dicyclohexylenemethane, n and z denote an integer ranging from 2 to 20, with n+z ranging from 4 to 10, E represents a group —O—R₃—O— in which R₃ represents a linear or branched C₁-C₁₅ alkylene radical such as methylene, propylene, butylene or ethylene, optionally interrupted with one or more heteroatoms, and r and s denote an integer ranging from 4 to 30.

Advantageously, the total amount of the (poly)carbodiimide compound(s) ranges from 0.01% to 40% by weight, preferably from 0.5% to 30% by weight, more preferentially from 1% to 20% by weight, even more preferentially from 2% to 15% and better still from 3% to 10% by weight relative to the total weight of composition C.

### Non-Carboxylic Anionic Thickener

Composition C may also comprise at least one anionic thickener, preferably a non-carboxylic anionic thickener.

For the purposes of the present invention, the term “non-carboxylic agent” means an agent which does not comprise any carboxylic acid functions (—COOH) or carboxylate functions (—COO—).

For the purposes of the present invention, the term “thickener” means a compound which increases the viscosity of a composition into which it is introduced to a concentration of 0.05% by weight relative to the total weight of the composition, by at least 20 cps, preferably by at least 50 cps, at room temperature (25° C.), at atmospheric pressure and at a shear rate of 1 s⁻¹ (the viscosity may be measured using a cone/plate viscometer, a Haake R600 rheometer or the like).

Preferably, the non-carboxylic anionic thickener(s) are chosen from non-carboxylic anionic polymers, more preferentially from anionic polymers bearing (a) sulfonic group(s).

For the purposes of the invention, the term “anionic polymer” means a polymer comprising one or more anionic or anionizable groups, and not comprising any cationic or cationizable groups.

Advantageously, the non-carboxylic anionic thickener(s) are chosen from anionic polymers including at least one ethylenically unsaturated monomer bearing a sulfonic group, in free form or partially or totally neutralized form.

These polymers may be crosslinked or non-crosslinked. They are preferably crosslinked.

These polymers may be associative or non-associative, preferably non-associative.

It is recalled that “associative polymers” are polymers that are capable, in an aqueous medium, of reversibly associating with each other or with other molecules.

Their chemical structure more particularly comprises at least one hydrophilic zone and at least one hydrophobic zone.

The term “hydrophobic group” means a radical or polymer with a saturated or unsaturated, linear or branched hydrocarbon-based chain, comprising at least 8 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

The ethylenically unsaturated monomers bearing a sulfonic group are notably chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids such as undecylacrylamidomethanesulfonic acid, and also partially or totally neutralized forms thereof.

(Meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof, will more preferentially be used. 2-Acrylamido-2-methylpropanesulfonic acid (AMPS), and also partially or totally neutralized forms thereof, will more particularly be used.

Among the 2-acrylamido-2-methylpropanesulfonic acid copolymers, mention may be made of partially or totally neutralized crosslinked copolymers of 2-acrylamido-2-methylpropanesulfonic acid and of acrylamide; mention may be made in particular of the product described in Example 1 of EP 503 853, and reference may be made to said document as regards these polymers.

Mention may also be made of copolymers of 2-acrylamido-2-methylpropanesulfonic acid or salts thereof and of hydroxyethyl acrylate, such as the compound sold under the name Sepinov EMT 10 by the company SEPPIC (INCI name: hydroxyethylacrylate/sodium acryloyldimethyl taurate copolymer).

The associative AMPS polymers may notably be chosen from statistical associative AMPS polymers modified by reaction with a C₆-C₂₂ n-monoalkylamine or di-n-alkylamine, and such as those described in patent application WO 00/31154 (forming an integral part of the content of the description). These polymers may also contain other ethylenically unsaturated hydrophilic monomers chosen, for example, from (meth)acrylic acid derivatives, such as esters thereof obtained with monoalcohols or mono- or polyalkylene glycols, (meth)acrylamides, vinylpyrrolidone, or mixtures of these compounds.

The preferred polymers of this family are chosen from associative copolymers of AMPS and of at least one ethylenically unsaturated hydrophobic monomer.

These same copolymers may also contain one or more ethylenically unsaturated monomers not including a fatty chain, such as (meth)acrylic acid derivatives, notably esters thereof obtained with monoalcohols or mono- or polyalkylene glycols, (meth)acrylamides, vinylpyrrolidone, or mixtures of these compounds.

These copolymers are described notably in patent application EP-A 750 899, patent U.S. Pat. No. 5,089,578 and in the following publications from Yotaro Morishima:
Self-assembling amphiphilic polyelectrolytes and their nanostructures, Chinese Journal of Polymer Science, Vol. 18, No. 40, (2000), 323-336;
Micelle formation of random copolymers of sodium 2-(acrylamido)-2-methylpropanesulfonate and a nonionic surfactant macromonomer in water as studied by fluorescence and dynamic light scattering, Macromolecules, Vol. 33, No. 10, (2000), 3694-3704;
Solution properties of micelle networks formed by non-ionic moieties covalently bound to an polyelectrolyte: salt effects on rheological behavior—Langmuir, Vol. 16, No. 12, (2000) 5324-5332;
Stimuli responsive amphiphilic copolymers of sodium 2-(acrylamido)-2-methylpropanesulfonate and associative macromonomers—Polym. Preprint, Div. Polym. Chem. 40(2), (1999), 220-221.

Among these polymers, mention may be made of:
crosslinked or non-crosslinked, neutralized or non-neutralized copolymers, including from 15% to 60% by weight of AMPS units and from 40% to 85% by weight of (C₈-C₁₆)alkyl(meth)acrylamide or (C₈-C₁₆)alkyl(meth)acrylate units relative to the polymer, such as those described in patent application EP-A750 899;
terpolymers including from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

Preferably, the non-carboxylic anionic thickener(s) are chosen from sodium 2-acrylamido-2-methylpropanesulfonate/hydroxyethyl acrylate copolymer, sold by the company SEPPIC (INCI name hydroxyethyl acrylate/sodium acryloyldimethyl taurate copolymer).

Advantageously, the total amount of the anionic thickener(s) can range from 0.01% to 20% by weight, preferably from 0.1% to 10% by weight, better still from 0.1% to 5% by weight, and even better still from 0.1% to 3% by weight, relative to the total weight of composition C.

Advantageously, the total amount of the non-carboxylic anionic thickener(s) can range from 0.01% to 20% by weight, preferably from 0.1% to 10% by weight, better still from 0.1% to 5% by weight, and even better still from 0.1% to 3% by weight, relative to the total weight of composition C.

### Amino Silicone

Composition C employed within the context of the process according to the invention may also comprise at least one amino silicone.

The term “amino silicone” denotes any silicone including at least one primary, secondary or tertiary amine or a quaternary ammonium group.

The weight-average molecular masses of these amino silicones may be measured by gel permeation chromatography (GPC) at room temperature (25° C.), as polystyrene equivalent. The columns used are p styragel columns. The eluent is THF and the flow rate is 1 ml/min. 200 μl of a 0.5% by weight solution of silicone in THF are injected. Detection is performed by refractometry and UV-metry.

Preferably, the amino silicone(s) that may be used in the context of the invention are chosen from:
a) the polysiloxanes corresponding to formula (A):

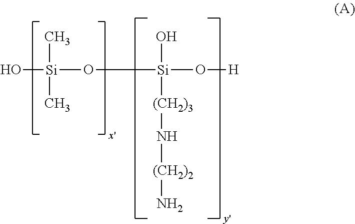

in which x′ and y′ are integers such that the weight-average molecular weight (Mw) is between 5000 and 500 000 approximately;
b) the amino silicones corresponding to formula (B):

R′ₐG₃₋ₐ-Si(OSiG₂)ₙ-(OSiG_{b}R′_{2-b})ₘ—O-SiG₃₋ₐ-R′ₐ (B)
in which:
   G, which may be identical or different, denotes a hydrogen atom or a group from among phenyl, OH, C₁-C₈ alkyl, for example methyl, or C₁-C₈ alkoxy, for example methoxy,
   a, which may be identical or different, denotes 0 or an integer from 1 to 3, in particular 0,
   b denotes 0 or 1, in particular 1,
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
   R′, which may be identical or different, denotes a monovalent radical of formula —CqH2qL in which q is a number ranging from 2 to 8 and L is an optionally quaternized amine group chosen from the following groups:
—N(R″)₂; —N⁺(R″)₃A-; —NR″-Q-N(R″)₂ and —NR″-Q-N⁺(R″)₃A-,
in which R″, which may be identical or different, denotes hydrogen, phenyl, benzyl, or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A- represents a cosmetically acceptable anion, notably a halide anion such as fluoride, chloride, bromide or iodide.

Preferably, the amino silicone(s) are chosen from the amino silicones of formula (B).

Preferably, the amino silicones of formula (B) are chosen from the amino silicones corresponding to formulae (C), (D), (E), (F) and/or (G) below.

According to a first embodiment, the amino silicones corresponding to formula (B) are chosen from the silicones known as “trimethylsilyl amodimethicone” corresponding to formula (C):

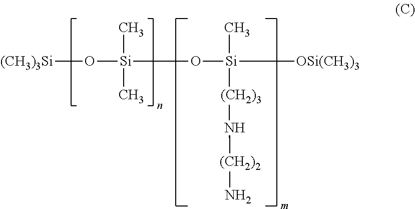

in which m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10.

According to a second embodiment, the amino silicones corresponding to formula (B) are chosen from the silicones of formula (D) below:

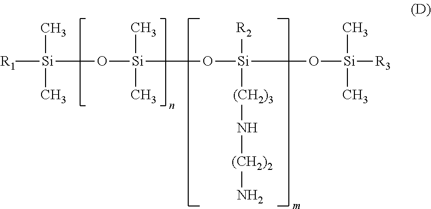

in which:
   m and n are numbers such that the sum (n+m) ranges from 1 to 1000, in particular from 50 to 250 and more particularly from 100 to 200; n possibly denoting a number from 0 to 999, notably from 49 to 249 and more particularly from 125 to 175, and m possibly denoting a number from 1 to 1000, notably from 1 to 10 and more particularly from 1 to 5;
   R₁, R₂ and R₃, which may be identical or different, represent a hydroxyl or C₁-C₄ alkoxy radical, at least one of the radicals R₁ to R₃ denoting an alkoxy radical.

Preferably, the alkoxy radical is a methoxy radical.

The hydroxy/alkoxy mole ratio preferably ranges from 0.2:1 to 0.4:1 and preferably from 0.25:1 to 0.35:1 and more particularly is equal to 0.3:1.

The weight-average molecular mass (Mw) of these silicones preferably ranges from 2000 to 1 000 000 and more particularly from 3500 to 200 000.

According to a third embodiment, the amino silicones corresponding to formula (B) are chosen from the silicones of formula (E) below:

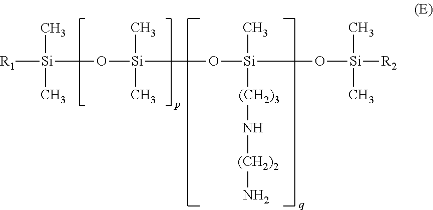

in which:
   p and q are numbers such that the sum (p+q) ranges from 1 to 1000, in particular from 50 to 350 and more particularly from 150 to 250; p possibly denoting a number from 0 to 999 and notably from 49 to 349 and more particularly from 159 to 239, and q possibly denoting a number from 1 to 1000, notably from 1 to 10 and more particularly from 1 to 5;
   R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly is equal to 1:0.95.

The weight-average molecular mass (Mw) of the silicone preferably ranges from 2000 to 200 000, even more particularly from 5000 to 100 000 and more particularly from 10 000 to 50 000.

The commercial products comprising silicones of structure (D) or (E) may include in their composition one or more other amino silicones the structure of which is different from formula (D) or (E).

A product containing amino silicones of structure (D) is sold by the company Wacker under the name Belsil® ADM 652.

A product containing amino silicones of structure (E) is sold by Wacker under the name Fluid WR 1300® or under the name Belsil ADM Log 1.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nm. Preferably, notably as amino silicones of formula (E), use is made of microemulsions with a mean particle size ranging from 5 nm to 60 nm (limits included) and more particularly from 10 nm to 50 nm (limits included). Thus, use may be made according to the invention of the amino silicone microemulsions of formula (E) sold under the names Finish CT 96 E® or SLM 28020® by the company Wacker.

According to a fourth embodiment, the amino silicones corresponding to formula (B) are chosen from the silicones of formula (F) below:

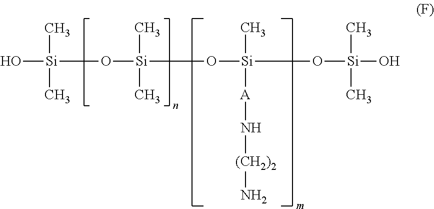

in which:
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
   A denotes a linear or branched alkylene radical containing from 4 to 8 carbon atoms and preferably 4 carbon atoms. This radical is preferably linear.

The weight-average molecular mass (Mw) of these amino silicones preferably ranges from 2000 to 1 000 000 and even more particularly from 3500 to 200 000.

Another silicone corresponding to formula (B) is, for example, the Xiameter MEM 8299 Emulsion from Dow Corning (INCI name: amodimethicone and trideceth-6 and cetrimonium chloride).

According to a fifth embodiment, the amino silicones corresponding to formula (B) are chosen from the silicones of formula (G) below:

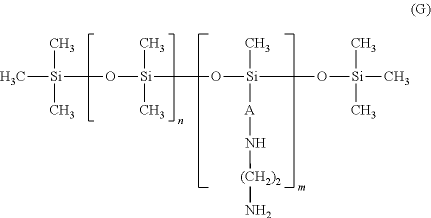

in which:
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
   A denotes a linear or branched alkylene radical containing from 4 to 8 carbon atoms and preferably 4 carbon atoms. This radical is preferably branched.

The weight-average molecular mass (Mw) of these amino silicones preferably ranges from 500 to 1 000 000 and even more particularly from 1000 to 200 000.

A silicone corresponding to this formula is, for example, DC2-8566 Amino Fluid from Dow Corning.
c) the amino silicones corresponding to formula (H):

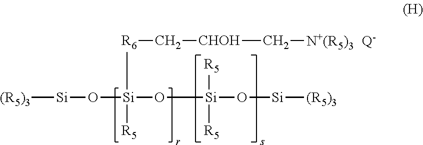

in which:
   R₅ represents a monovalent hydrocarbon-based radical containing from 1 to 18 carbon atoms, and in particular a C₁-C₁₈ alkyl or C₂-C₁₅ alkenyl radical, for example methyl;
   R₆ represents a divalent hydrocarbon-based radical, in particular a C₁-C₁₅ alkylene radical or a divalent C₁-C₁₅, for example C₁-C₈, alkyleneoxy radical linked to the Si via an SiC bond;
   Q⁻ is an anion, such as a halide ion, in particular a chloride ion, or an organic acid salt, in particular an acetate;
   r represents a mean statistical value ranging from 2 to 20 and in particular from 2 to 8;
   s represents a mean statistical value ranging from 20 to 200 and in particular from 20 to 50.

Such amino silicones are notably described in patent U.S. Pat. No. 4,185,087.
d) the quaternary ammonium silicones of formula (I):

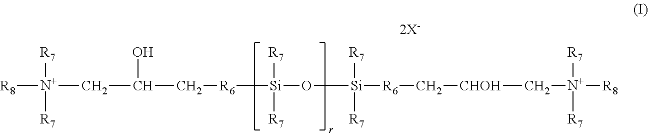

in which:
   R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example methyl;
   R₆ represents a divalent hydrocarbon-based radical, in particular a C₁-C₁₅ alkylene radical or a divalent C₁-C₁₅, for example C₁-C₈, alkyleneoxy radical linked to the Si via an SiC bond;
   R₈, which may be identical or different, represent a hydrogen atom, a monovalent hydrocarbon-based radical having from 1 to 18 carbon atoms, and in particular a C₁-C₁₈ alkyl radical, a C₂-C₁₅ alkenyl radical or a radical —R₆—NHCOR₇;
   X⁻ is an anion such as a halide ion, notably chloride, or an organic acid salt, notably acetate;
   r represents a mean statistical value ranging from 2 to 200 and in particular from 5 to 100.

These silicones are described, for example, in patent application EP-A 0 530 974.
e) the amino silicones of formula (J):

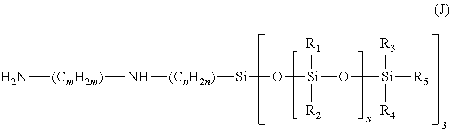

in which:
   R₁, R₂, R₃ and R₄, which may be identical or different, denote a C₁-C₄ alkyl radical or a phenyl group,
   R₅ denotes a C₁-C₄ alkyl radical or a hydroxyl group,
   n is an integer ranging from 1 to 5,
   m is an integer ranging from 1 to 5, and
   x is chosen such that the amine number ranges from 0.01 to 1 meq/g.
f) multiblock polyoxyalkylene amino silicones, of the type (AB)ₙ, A being a polysiloxane block and B being a polyoxyalkylene block including at least one amine group.

Said silicones are preferably formed from repeating units having the following general formulae:

[—(SiMe₂O)ₓSiMe₂-R—N(R″)—R′—O(C₂H₄O)ₐ(C₃H₆O)_{b}—R′—N(H)—R—]

or alternatively

[—(SiMe₂O)ₓSiMe₂-R—N(R″)—R′—O(C₂H₄O)ₐ(C₃H₆O)_{b}—]
in which:
   a is an integer greater than or equal to 1, preferably ranging from 5 to 200 and more particularly ranging from 10 to 100;
   b is an integer between 0 and 200, preferably ranging from 4 to 100 and more particularly between 5 and 30;
   x is an integer ranging from 1 to 10 000 and more particularly from 10 to 5000;
   R″ is a hydrogen atom or a methyl;
   R, which may be identical or different, represent a linear or branched divalent C₂-C₁₂ hydrocarbon-based radical, optionally including one or more heteroatoms such as oxygen; preferably, R denotes an ethylene radical, a linear or branched propylene radical, a linear or branched butylene radical or a CH₂CH₂CH₂OCH₂CH(OH)CH₂— radical; preferentially, R denotes a CH₂CH₂CH₂OCH₂CH(OH)CH₂— radical;
   R′, which may be identical or different, represent a linear or branched divalent C₂-C₁₂ hydrocarbon-based radical, optionally including one or more heteroatoms such as oxygen; preferably, R′ denotes an ethylene radical, a linear or branched propylene radical, a linear or branched butylene radical or a radical CH₂CH₂CH₂OCH₂CH(OH)CH₂—; preferentially, R′ denotes —CH(CH₃)—CH₂—.

The siloxane blocks preferably represent between 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1 000 000 and more particularly between 10 000 and 200 000.

Mention may notably be made of the silicones sold under the name Silsoft A− 843 or Silsoft A+ by Momentive.
g) and mixtures thereof.

Preferably, the amino silicones of formula (B) are chosen from the amino silicones corresponding to formula (E).

Preferably, composition C employed within the context of the process according to the invention comprises at least one amino silicone having the INCI name amodimethicone, preferably introduced in the form of an emulsion or microemulsion with surfactants.

Preferably, composition C employed within the context of the process according to the invention comprises at least one amino silicone having the INCI name amodimethicone as an emulsion or microemulsion with surfactants, having the INCI name trideceth-5 and trideceth-10.

The amino silicone(s) may be present in a total amount ranging from 0.01% to 20%, preferably from 0.05% to 15%, more preferentially from 0.1% to 10%, even more preferentially from 0.25% to 8% by weight and better still from 0.3% to 5% by weight relative to the total weight of composition C.

### Solvents

Composition C employed within the context of the process according to the invention may comprise one or more organic solvents.

Preferably, the composition comprises ethanol.

The organic solvents may be present in a total amount ranging from 0.01% to 50% by weight, preferably ranging from 0.1% to 45% by weight and preferably ranging from 1% to 40% by weight relative to the total weight of composition C.

Composition C employed within the context of the process according to the invention may be aqueous. The water content may range from 1% to 90% by weight, preferably from 10% to 80% by weight and more preferentially from 20% to 75% by weight relative to the total weight of composition C.

### Composition D:

Composition D employed within the context of the process according to the invention comprises at least one silicone elastomer containing carboxylic acid functions.

### Silicone Elastomer Containing Carboxylic Acid Functions

Composition D according to the invention comprises at least one silicone elastomer bearing carboxylic acid functions.

The term “organopolysiloxane elastomer” means a flexible, deformable organopolysiloxane having viscoelastic properties and notably the consistency of a sponge or a flexible sphere. Its modulus of elasticity is such that this material resists deformation and has a limited capacity for extension and contraction. This material is able to return to its original shape after stretching. In general, it is partially or totally crosslinked and is non-cyclic.

The term “silicone elastomer bearing carboxylic acid functions” means an elastomeric organopolysiloxane comprising in its structure at least one, preferably at least two, carboxylic acid groups with at least one carboxylic acid (—COOH) or carboxylate (—COO—) function.

Preferably, the silicone elastomer bearing carboxylic acid functions is partially or totally crosslinked.

According to this embodiment, the silicone elastomer bearing carboxylic acid functions is advantageously the product of reaction of a first siloxane, a second siloxane and a crosslinking agent.

The composition according to the invention advantageously has a total content of silicone elastomer(s) bearing carboxylic acid functions ranging from 0.1% to 40% by weight relative to the weight of composition D. Preferably, this content ranges from 1% to 35% by weight, more preferentially from 5% to 30% by weight, better still from 10% to 25% by weight relative to the weight of composition D.

The composition according to the invention advantageously has a total content of crosslinked silicone elastomer(s) bearing carboxylic acid functions ranging from 0.1% to 40% by weight relative to the weight of composition D. Preferably, this content ranges from 1% to 35% by weight, more preferentially from 5% to 30% by weight, better still from 10% to 25% by weight relative to the weight of composition D.

The silicone elastomers bearing carboxylic acid functions that may be used in the compositions of the invention may be chosen from those described in the published documents WO 2015/066161 (US 2016/0200876), WO 2015/066165, WO 2015/066199, WO 2015/167963, WO 2018/165434, (U.S. Ser. No. 10/918,587B2), IPCOM000250448D and IPCOM000250657D.

Preferably, the silicone elastomer bearing carboxylic acid functions is chosen from those corresponding to formula (XIII) below:

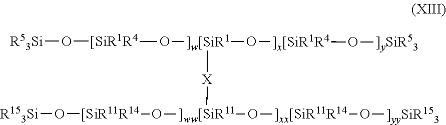

in which:
the radical X corresponds to formula (i) below:

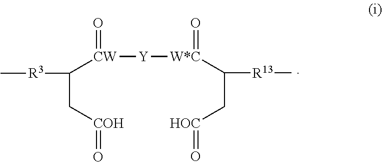

or to formula (i′) below:

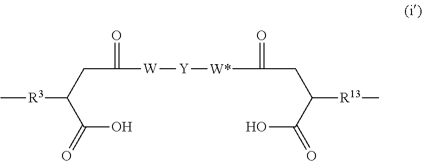

in which:
W and W* independently denote an oxygen atom (O) or a group N—R in which each radical R independently denotes a hydrogen atom (H) or a radical R¹;
Y is a divalent group;
R¹, R¹¹, R⁴, R¹⁴, R⁵ and R¹⁵ independently denote a linear or branched, saturated or unsaturated, cyclic or acyclic, substituted or unsubstituted hydrocarbon-based radical;
R³ and R¹³ independently denote a divalent group;
w and ww are independently integers ranging from 0 to 1000.
x and xx are independently integers ranging from 1 to 100;
y and yy are independently integers ranging from 0 to 1000;
preferably, w and y are not simultaneously equal to 0 and ww and yy are not simultaneously equal to 0.

The term “substituted” means that one or more hydrogen atoms of the hydrocarbon-based radical are replaced with an atom other than hydrogen (i.e. halogen) or that one or more carbon atoms are replaced with an atom other than carbon, such as a heteroatom, for instance oxygen, sulfur or nitrogen.

In formula (XIII), the lower and upper portions are identical or different siloxanes. According to certain particular modes, each siloxane chain comprises siloxane (Si—O—Si) bonds on their respective backbone.

In various embodiments, each siloxane chain may include siloxane bonds separated by one or more divalent groups, for example a —CH₂— bonding group. Other examples of divalent groups may include polyether groups: e.g. —CH₂CH₂O— bonding groups (i.e., ethylene oxide group), —CH(CH₃)CH₂O— (i.e., propylene oxide), etc.

Combinations of different divalent groups may be present on their respective backbones. Each of the divalent groups may be unique or repeated i.e.: 2 times, 5 times, 10 times, or even more than 10 times, etc. According to certain embodiments, the siloxanes do not comprise a polyether group.

In various embodiments, one or both siloxanes may comprise at least one [SiR¹R²—O—] unit (“D” units or R*₂SiO_{2/2}). Typically, each siloxane has a repetition of D units, which generally constitutes the linear portions of the siloxanes. The siloxanes also typically have terminal R*₃SiO_{1/2} units (“M” units).

In certain particular modes, one or both of the siloxanes may optionally be branched, partially branched and/or include a resin portion having a network of three-dimensional structure. In such cases, the corresponding siloxane(s) may also comprise R*SiO_{3/2} units (“T” units) and/or SiO_{4/2} units (“Q” units). The branched or resinous nature of the siloxane(s) may be attributed to the presence of T and/or Q units. The branched nature of a siloxane may be attributed to side groups of one or more D units. According to particular modes, one or both siloxanes may be free of T units and/or Q units. The two siloxanes may be identical or different: i.e. one is linear and the other branched, or both siloxanes are linear.

According to certain particular modes, the radicals R¹ in the compounds of formula (XIII) independently denote an alkyl, aryl, alkenyl, alkaryl or aralkyl radical.

More particularly, the radicals R¹ denote an alkyl radical, preferably containing from 1 to 20, from 1 to 15, from 1 to 10, from 1 to 6, from 1 to 4, or 1 or 2 carbon atoms, for instance methyl, ethyl, propyl, butyl or pentyl.

More particularly, all the radicals R¹ denote a methyl group (i.e. —CH₃).

Preferably, the radical R³ is a divalent, saturated or unsaturated hydrocarbon-based radical, optionally substituted with at least one heteroatom such as oxygen, sulfur or nitrogen. In particular, R³ is a group (CH₂)ₙ where n is an integer ranging from 1 to 30, from 1 to 25, from 1 to 20, from 1 to 15, from 1 to 10, from 1 to 5, or from 1 to 3, and more particularly equal to 3.

According to various particular modes, each of the radicals R⁴ and R⁵ may independently denote a substituted or unsubstituted hydrocarbon-based radical, notably chosen from those defined previously for the radicals R¹.

According to certain modes, each radical R⁴ independently denotes an alkyl or aryl (i.e. phenyl) group or a group (R⁶O)ₘ.

When R⁴ is a group (R⁶O)ₘ, R⁶ is preferably an alkyl or aryl (i.e.: phenyl) radical and m is an integer ranging from 1 to 50, from 1 to 25, from 1 to 10, from 1 to 5, or 1. The group (R⁶O)ₘ may be a polyether group (i.e.: with ethylene oxide and/or propylene oxide units). According to particular modes, a radical R⁴ may be a divalent group of a silicone side chain of the siloxane.

According to a particular mode, R⁴ may denote an anhydride group of formula (A)

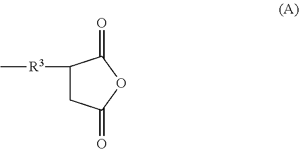

in which R³ has the identical definition indicated previously.

According to a particular mode, R⁴ may denote an alkyl radical, preferably hexyl.

Preferably, the radicals R⁴ denote an alkyl radical or a polyether group.

Preferably, the radicals R⁵ independently denote an alkyl, aryl, alkenyl, alkaryl, or aralkyl radical.

More particularly, the radicals R⁵ denote an alkyl radical, preferably containing from 1 to 20, from 1 to 15, from 1 to 10, from 1 to 6, from 1 to 4, or 1 or 2 carbon atoms, for instance methyl, ethyl, propyl, butyl or pentyl.

More particularly, all the radicals R⁵ denote a methyl group (i.e. —CH₃).

Preferably, w is an integer ranging from 0 to 1000, from 0 to 950, from 0 to 750, from 0 to 500, from 0 to 400, from 1 to 350, from 1 to 300, from 25 to 250, from 50 to 200, from 50 to 150, from 75 to 125, from 90 to 110, from 90 to 100, from 90 to 95, and most particularly is equal to 93.

Preferably, x is an integer from 1 to 100, from 1 to 75, from 1 to 50, from 1 to 25, from 1 to 20, from 1 to 10, or from 1 to 5, and more particularly equal to 3.

Preferably, y is an integer ranging from 0 to 1000, from 0 to 950, from 0 to 750, from 0 to 500, from 0 to 400, from 1 to 350, from 1 to 300, from 1 to 250, from 1 to 200, from 1 to 150, from 1 to 100, from 1 to 75, from 1 to 50, from 1 to 25, from 1 to 20, from 1 to 15, from 1 to 10, or from 1 to 5.

According to various embodiments, w and y are not simultaneously equal to 0. In certain modes, the sum w+x+y ranges from 25 to 1500, from 25 to 1000, from 25 to 900, from 25 to 800, from 25 to 700, from 25 to 600, from 25 to 500, from 25 to 400, from 25 to 300, from 50 to 200, from 75 to 150, from 85 to 125 or from 90 to 110.

In certain modes, x is at least equal to 1, at least equal to 10, at least equal to 25, at least equal to 50, at least equal to 75 or at least equal to 5.

The units of formula (XIII), notably those represented in square brackets indexed by w, ww, x, xx, y or yy, may be present in any order, randomly or in sequential form.

According to certain particular modes, the radicals R¹¹, which may be identical or different, denote a linear, branched or cyclic C₁-C₂₀, C₁-C₁₅, C₁-C₁₀, C₁-C₆, C₁-C₄ or C₁-C₂ alkyl radical such as methyl, ethyl, propyl, butyl, pentyl groups, etc. More preferentially, the radicals R¹¹ are a methyl group.

According to certain particular modes, the radicals R¹⁴, which may be identical or different, denote an alkyl, aryl (i.e. phenyl) or (R¹⁶O)ₘₘ radical. When R¹⁴ is a group (R¹⁶O)ₘₘ, R¹⁶ denotes an alkyl or aryl (i.e. phenyl) group and mm is an integer ranging from 1 to 50, 1 to 25, 1 to 10, 1 to 5, or 1. The group (R¹⁶O)ₘₘ may be a polyether group (i.e. with ethylene oxide and/or propylene oxide units).

According to particular modes, the radicals R¹⁴ independently denote an alkyl radical containing 2 to 20, 2 to 15, 2 to 10, 2 to 5, or 2 carbon atoms.

In other particular modes, R¹⁴ may be a divalent bonding group of a silicone side chain of the siloxane.

According to a particular mode, R¹⁴ may denote an anhydride group of formula (A) as defined previously.

According to a particular mode, R¹⁴ may denote an alkyl radical, preferably hexyl.

Preferably, the radicals R¹⁴ denote an alkyl radical or a polyether group.

Preferably, the radicals R¹⁵ independently denote an alkyl, aryl, alkenyl, alkaryl, or aralkyl radical.

More particularly, the radicals R¹⁵ denote an alkyl radical, preferably containing from 1 to 20, from 1 to 15, from 1 to 10, from 1 to 6, from 1 to 4, or 1 or 2 carbon atoms, for instance methyl, ethyl, propyl, butyl or pentyl.

More particularly, all the radicals R¹⁵ denote a methyl group (i.e. —CH₃).

Preferably, ww is an integer ranging from 0 to 1000, from 0 to 950, from 0 to 750, from 0 to 500, from 0 to 400, from 1 to 350, from 1 to 300, from 25 to 250, from 50 to 200, from 50 to 150, from 75 to 125, from 90 to 110, from 90 to 100, from 90 to 95, and most particularly 93.

Preferably, xx is an integer ranging from 1 to 100, from 1 to 75, from 1 to 50, from 1 to 25, from 1 to 20, from 1 to 10 or from 1 to 5, and more particularly 3.

Preferably, yy is an integer ranging from 0 to 1000, from 0 to 950, from 0 to 750, from 0 to 500, from 0 to 400, from 1 to 350, from 1 to 300, from 1 to 250, from 1 to 200, from 1 to 150, from 1 to 100, from 1 to 75, from 1 to 50, from 1 to 25, from 1 to 20, from 1 to 15, from 1 to 10 or from 1 to 5.

In various embodiments, ww and yy are not simultaneously equal to 0. According to certain particular modes, the sum ww+xx+yy ranges from 25 to 1500, from 25 to 1000, from 25 to 900, from 25 to 800, from 25 to 700, from 25 to 600, from 25 to 500, from 25 to 400, from 25 to 300, from 50 to 200, from 75 to 150, from 85 to 125, or from 90 to 110.

According to certain particular modes, xx is at least equal to 1, at least equal to 10, at least equal to 25, at least equal to 50, at least equal to 75, or at least equal to 85.

The radical X of formula (XIII) corresponds to the general formula (i) below:

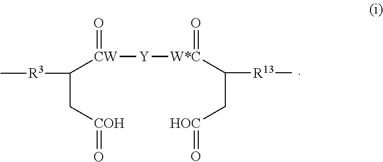

or to the general formula (i′) below:

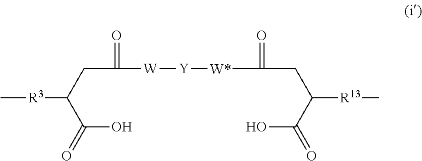

in which:
W and W* independently denote an oxygen atom (O) or a group N—R, with R denoting a hydrogen atom (H) or a radical R¹; preferably, R denotes a hydrogen atom;
   R³ is a divalent, saturated or unsaturated hydrocarbon-based radical, optionally substituted with at least one heteroatom such as oxygen, sulfur or nitrogen; preferably, R³ denotes a radical (CH₂)ₙ where n is an integer from 1 to 30, from 1 to 20, from 1 to 15, from 1 to 10, from 1 to 5, from 1 to 3, and more particularly 3;
   R¹³ is a divalent, saturated or unsaturated hydrocarbon-based radical, optionally substituted with at least one heteroatom such as oxygen, sulfur or nitrogen; preferably, R¹³ denotes a radical (CH₂)ₙ where n is an integer from 1 to 30, from 1 to 20, from 1 to 15, from 1 to 10, from 1 to 5, from 1 to 3, and more particularly 3;
   Y denotes a divalent, saturated or unsaturated hydrocarbon-based radical, optionally substituted with at least one heteroatom such as oxygen, sulfur or nitrogen; preferably, Y is a hydrocarbon-based radical containing from 1 to 50, from 1 to 40, from 1 to 20, from 1 to 10, from 1 to 5, 1 or 2 carbon atoms or denotes a group of formula (ii) below:

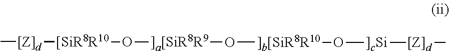

in which:
R⁸ and R⁹ independently denote a saturated or unsaturated, cyclic or acyclic, substituted or unsubstituted hydrocarbon-based radical, preferably chosen from an alkyl group (i.e. methyl), an aryl group (i.e. phenyl), a polyether group (i.e. with ethylene oxide and/or propylene oxide units);
   R¹⁰ independently denotes a saturated or unsaturated, cyclic or acyclic, substituted or unsubstituted hydrocarbon-based radical such as the radicals defined previously for R¹ and R⁴; preferably, R¹⁰ is an alkyl group containing from 1 to 20 carbon atoms or a polyether group or a divalent bonding group of a silicone side chain of the siloxane;
   Z independently denotes a divalent, saturated or unsaturated hydrocarbon-based radical, optionally substituted with at least one heteroatom such as oxygen, sulfur or nitrogen; preferably, Z is a hydrocarbon-based radical containing from 1 to 20, from 1 to 10, from 1 to 5, or 1 carbon atom(s); notably, Z may include siloxane bonds separated by one or more divalent groups, for example a —CH₂— bonding group; further examples of divalent groups may include polyether groups: for example —CH₂CH₂O— bonding groups (i.e. ethylene oxide group), —CH(CH₃)CH₂O— (i.e. propylene oxide);
   a is an integer ranging from 0 to 1000, from 0 to 950, from 0 to 750, from 0 to 500, from 0 to 400, from 0 to 300, from 0 to 200, from 0 to 100, from 0 to 75, from 0 to 50, from 0 to 25, or from 0 to 20;
   b is an integer ranging from 1 to 1000, from 1 to 950, from 1 to 750, from 1 to 500, from 1 to 400, from 1 to 300, or from 1 to 200;
   c is an integer ranging from 0 to 1000, from 0 to 950, from 0 to 750, from 0 to 500, from 0 to 400, from 0 to 300, from 0 to 200, or from 0 to 100;
   d is equal to 0 or 1, preferably equal to 1.

The units of formula (ii) represented in square brackets indexed by a, b, or c are present in any order, randomly or in sequential form.

According to a particular form, the siloxane of formula (ii) may be a resin of formula R*ₛSiO_{(4-s)/2}. Preferably, a silicone resin has along its chain T and/or Q units with M units and optionally D units. The radicals R* may be independently chosen from a substituted (i.e. hydroxyl, amine functions) or unsubstituted hydrocarbon-based radical and s ranges from 0 to 3. The groups R* that may be used may be those described previously for the radicals R⁸, R⁹ and R¹⁰. Various combinations of such groups may be present. According to these particular cases, the silicone resin comprises a combination of M, D, T and/or Q units. It may notably be an MDT resin, an MT resin, an MDQ resin, an MQ resin or an MDTQ resin. Each of the M, D and T units may contain different groups R*. The resin may have various molecular weights such as a number-average molecular weight ranging from 800 to 500 000.

According to a particular form of the invention, the silicone elastomers bearing carboxylic acid functions have a carboxyl equivalent ranging from 100 to 50 000, from 500 to 10 000 or from 500 to 5000.

The silicone elastomers bearing carboxylic functions of formula (XIII) may be obtained according to one of the synthetic processes described in WO 2015/066161 (US 2016/0200876).

According to a first variant of the synthetic process, certain silicone elastomers bearing carboxylic acid functions may be obtained by reacting 1) a first siloxane bearing anhydride side groups, 2) a second siloxane bearing anhydride side groups and 3) an organic polyol. According to a particular mode, one or more siloxanes different from the first two may be reacted with the three preceding reagents.

According to a second variant of the synthetic process, certain silicone elastomers bearing carboxylic acid functions may be obtained by reacting 1) a first siloxane bearing anhydride side groups, 2) a second siloxane bearing anhydride side groups and 3) an organic polyamine. According to a particular mode, one or more additional siloxanes different from the first two may be reacted with the three preceding reagents.

According to a third variant of the synthetic process, certain silicone elastomers bearing carboxylic acid functions may be obtained by reacting 1) a first siloxane bearing anhydride side groups, 2) a second siloxane bearing anhydride side groups and 3) a third siloxane containing at least two hydroxyl groups. According to a particular mode, one or more additional siloxanes different from the first two may be reacted with the three preceding reagents.

According to a fourth variant of the synthetic process, certain silicone elastomers bearing carboxylic acid functions may be obtained by reacting 1) a first siloxane bearing anhydride side groups, 2) a second siloxane bearing anhydride side groups and 3) a third siloxane containing amine side groups and/or amine end groups. According to a particular mode, one or more additional siloxanes different from the first two may be reacted with the three preceding reagents.

According to a fifth variant of the synthetic process, certain silicone elastomers bearing carboxylic acid functions may be obtained by reacting 1) a first siloxane bearing hydroxyl side groups, 2) a second siloxane bearing hydroxyl side groups and 3) a third siloxane containing at least two anhydride end groups. According to a particular mode, one or more additional siloxanes different from the first two may be reacted with the three preceding reagents.

According to a sixth variant of the synthetic process, certain silicone elastomers bearing carboxylic acid functions may be obtained by reacting 1) a first siloxane bearing amine side groups, 2) a second siloxane bearing amine side groups and 3) a third siloxane containing at least two anhydride end groups. According to a particular mode, one or more additional siloxanes different from the first two may be reacted with the three preceding reagents.

According to a seventh variant of the synthetic process, certain silicone elastomers bearing carboxylic acid functions may be obtained by reacting 1) a first organic alcohol, 2) a second organic alcohol and 3) a siloxane containing anhydride end groups. According to a particular mode, one or more additional organic alcohols different from the first two may be reacted with the three preceding reagents.

Among the silicone elastomers bearing carboxylic acid functions of formula (XIII), mention may be made of compounds 1 to 12 below as described, respectively, in Examples 4, 5 and 7 to 16 of patent application WO 2015/066161 or in the examples of patent application US 2020/0323765:

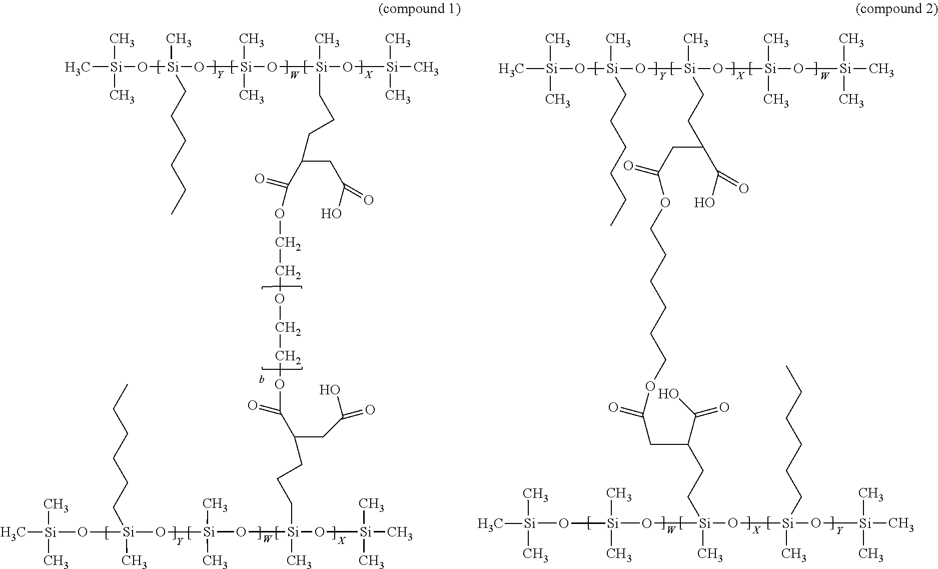

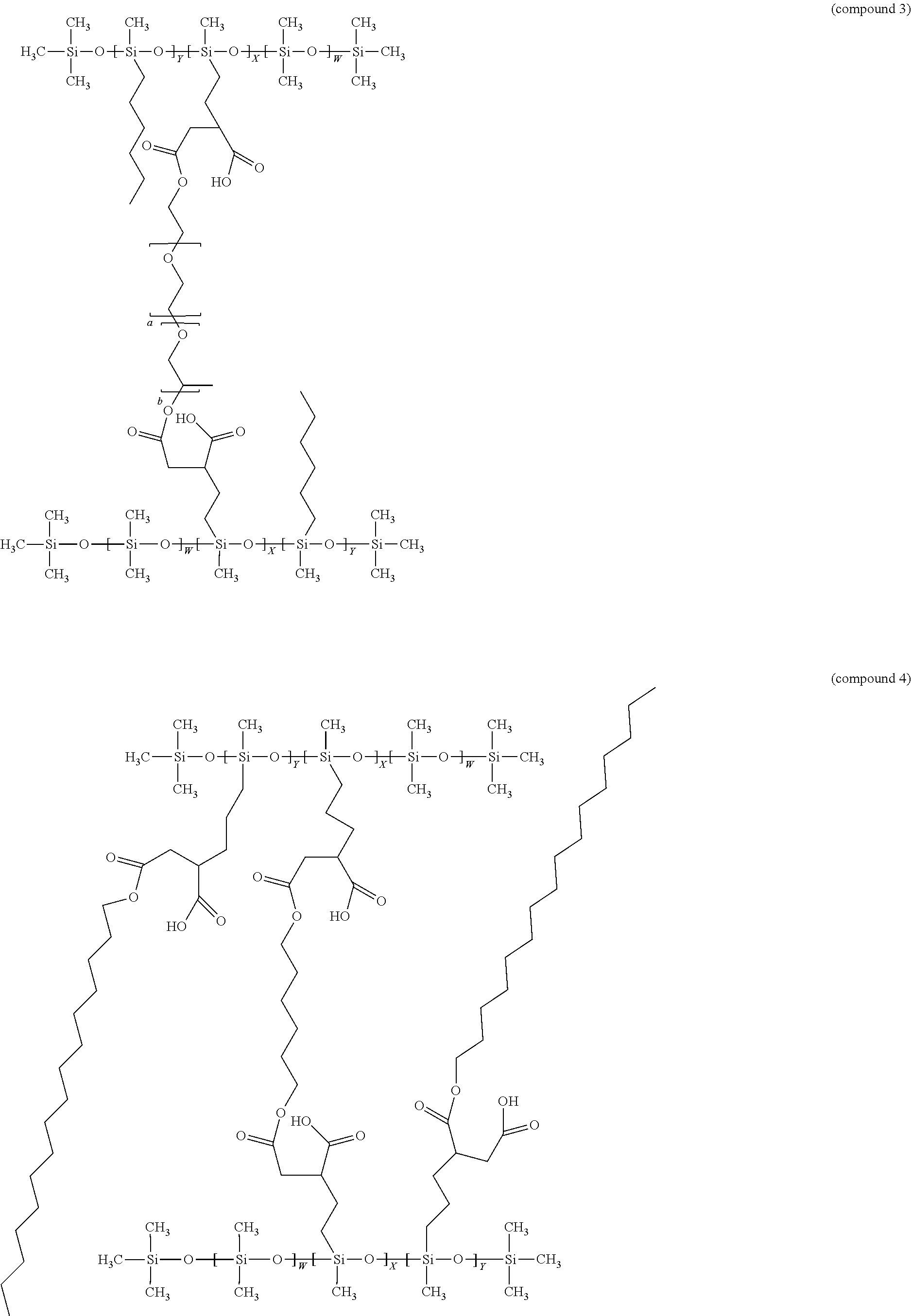

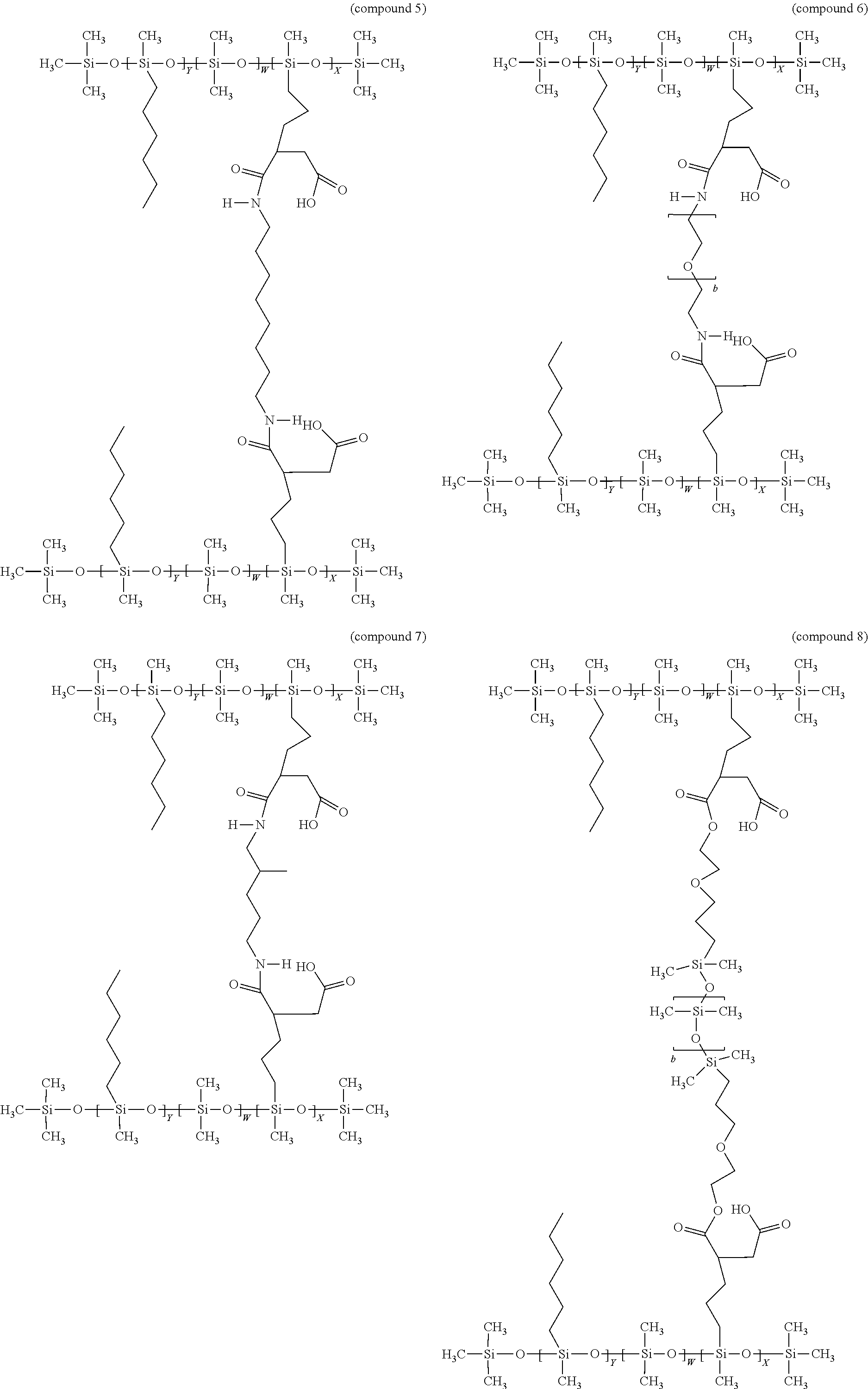

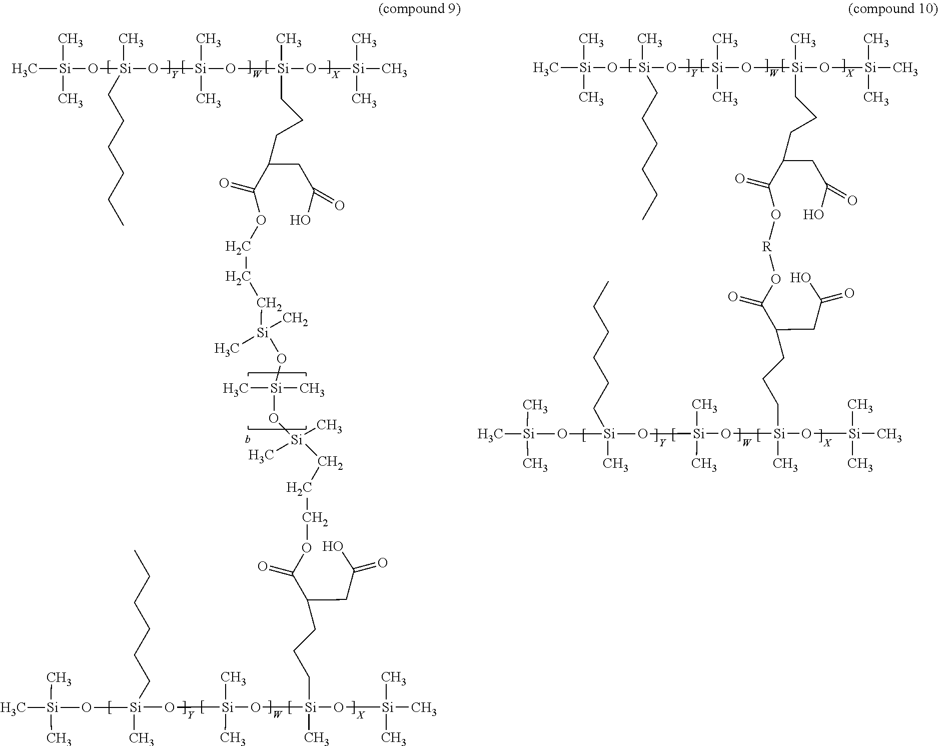

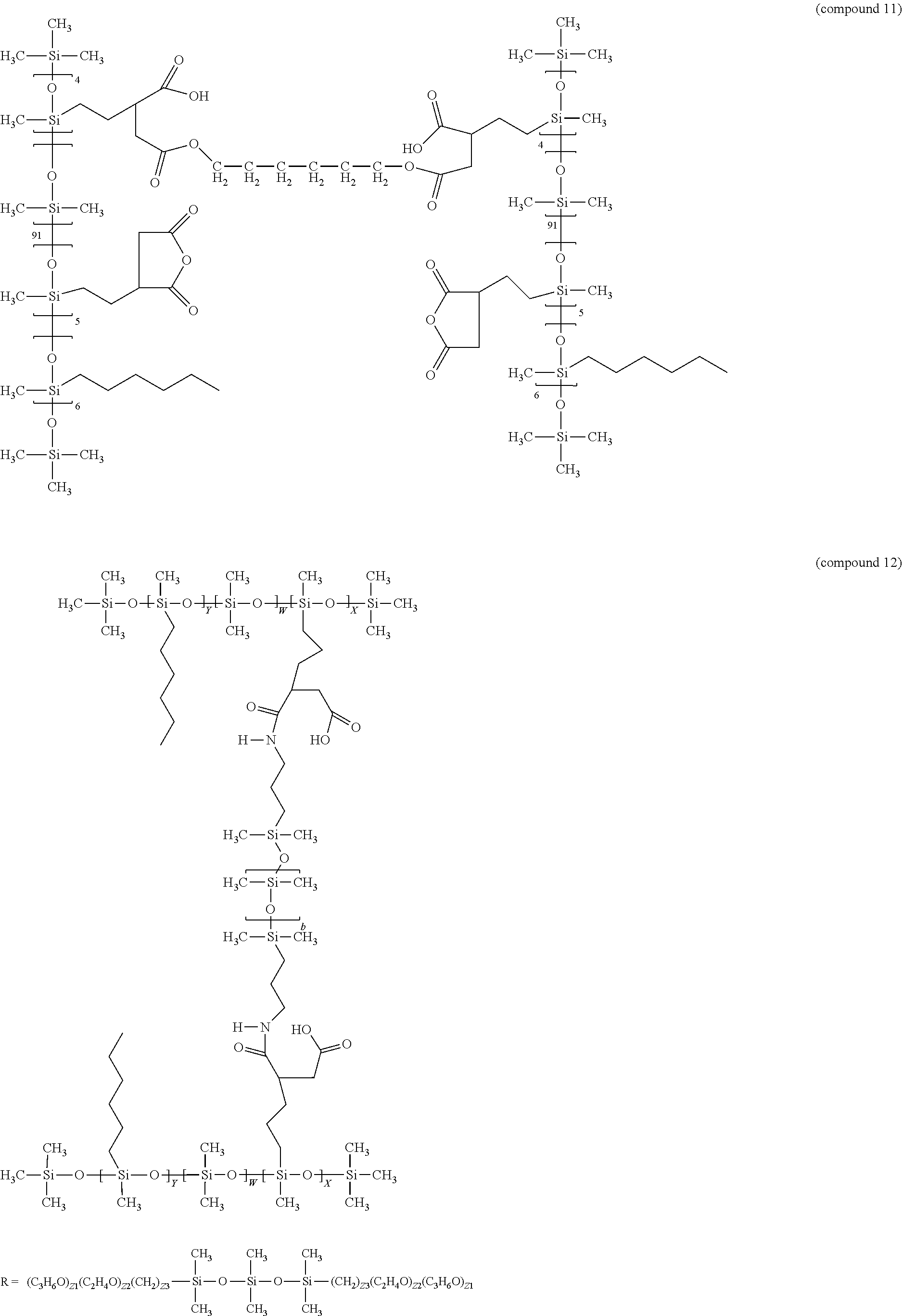

According to a particular form, the composition of the invention contains at least compound 11 as defined previously.

Among the silicone elastomers bearing carboxylic acid functions that may be used in the compositions of the invention, mention may also be made of the silicone elastomer having the INCI name: Hexyl/Succinyl Dimethicone Crosspolymer.

According to a particular form, the silicone elastomers bearing carboxylic acid functions are in solid form. They may notably be chosen from compounds 2 and 3 as defined previously.

According to another particular form, the silicone elastomers bearing carboxylic acid functions are in dispersion in at least one oil and are preferably in gel form.

The term “oil” means a fatty substance which is liquid at room temperature (25° C.) and atmospheric pressure (760 mmHg or 10⁵ Pa).

The oil may be chosen from volatile oils and/or non-volatile oils.

The term “non-volatile oil” means an oil which remains on the keratin material at room temperature (25° C.) and atmospheric pressure (760 mmHg) for at least several hours and which notably has a vapour pressure of less than 10 mmHg (0.13 Pa).

The oil may be chosen from hydrocarbon-based oils.

The term “hydrocarbon-based oil” means an oil mainly including carbon and hydrogen atoms and optionally one or more functions chosen from hydroxyl, ester, ether and carboxylic functions.

As examples of volatile hydrocarbon-based oils that may be used in the oily dispersion of a silicone elastomer bearing carboxylic functions, mention may be made of hydrocarbon-based oils containing from 8 to 16 carbon atoms, and notably C₈-C₁₆ isoalkanes of petroleum origin (also called isoparaffins) such as isododecane (also called 2,2,4,4,6-pentamethylheptane), isodecane, isohexadecane, and for example oils sold under the trade names Isopars or Permethyls, branched C₈-C₁₆ esters, isohexyl neopentanoate, and mixtures thereof. Other volatile hydrocarbon-based oils such as petroleum distillates, notably those sold under the name Shell Solt by the company Shell, may also be used; volatile linear alkanes such as those described in the patent application of the company Cognis DE10 2008 012 457.

As examples of linear non-volatile hydrocarbon-based oils, mention may be made of:
synthetic esters, such as the oils of formula R₁COOR₂, in which R₁ represents a residue of a linear or branched fatty acid containing from 1 to 40 carbon atoms, and R₂ represents a hydrocarbon-based chain, notably a branched chain containing from 1 to 40 carbon atoms, provided that the sum of the number of carbon atoms in the chains R₁ and R₂ is greater than or equal to 10; the esters may be chosen, notably, from esters of fatty alcohol and fatty acid, for instance cetostearyl octanoate, esters of isopropyl alcohol, such as isopropyl myristate, isopropyl palmitate, ethyl palmitate, 2-ethylhexyl palmitate, isopropyl stearate, isopropyl isostearate, isostearyl isostearate, octyl stearate, hydroxy esters, such as isostearyl lactate, octyl hydroxystearate, diisopropyl adipate, heptanoates, and notably isostearyl heptanoate, octanoates, decanoates or ricinoleates of alcohols or polyalcohols, such as propylene glycol dioctanoate, cetyl octanoate, tridecyl octanoate, 2-ethylhexyl 4-diheptanoate, 2-ethylhexyl palmitate, alkyl benzoate, polyethylene glycol diheptanoate, propylene glycol bis(2-ethylhexanoate), and mixtures thereof, benzoates of C₁₂-C₁₅ alcohols, hexyl laurate, neopentanoic acid esters, such as isodecyl neopentanoate, isotridecyl neopentanoate, isostearyl neopentanoate, octyldocecyl neopentanoate, isononanoic acid esters, such as isononyl isononanoate, isotridecyl isononanoate, octyl isononanoate, hydroxy esters such as isostearyl lactate, diisostearyl malate; and more particularly isodecyl neopentanoate;
dialkyl carbonates, the two alkyl chains possibly being identical or different, such as dicaprylyl carbonate sold under the name Cetiol CC® by Cognis;
synthetic ethers containing from 10 to 40 carbon atoms, such as dicaprylyl ether.

Preferably, the hydrocarbon-based oil used is isododecane.

Preferably, the oily dispersion of silicone elastomer bearing carboxylic acid functions comprises from 10% to 40% by weight of silicone elastomer active material, and more preferentially from 20% to 35% by weight of silicone elastomer active material relative to the total weight of the oily dispersion.

Preferably, the composition according to the invention comprises at least one silicone elastomer bearing carboxylic acid functions in dispersion in isododecane and preferably in gel form. In particular, said silicone elastomer is chosen from compounds 1 and 4 to 11 as defined previously.

More preferably, the composition according to the invention comprises compound 11 in dispersion in isododecane and in gel form, and more particularly in an active material content equal to 30% by weight relative to the total weight of the dispersion.

According to another particularly preferred form, the composition of the invention comprises a silicone elastomer bearing carboxylic acid functions in dispersion in isododecane and in gel form, having the INCI name: Isododecane (and) Hexyl/Succinyl Dimethicone Crosspolymer and more particularly in an active material content equal to 30% by weight relative to the total weight of the dispersion.

Mention may be made of the commercial product Dowsil EL-7314 Silicone Elastomer Blend® sold by the company Dow Corning, comprising 30% by weight of active material of said silicone elastomer.

### Colouring Agent

Composition C and/or composition D employed within the context of the process according to the invention comprise(s) one or more colouring agent(s) chosen from pigments, direct dyes and mixtures thereof.

Preferably, composition C and/or composition D employed within the context of the process according to the invention comprise(s) one or more pigments.

The term “pigment” refers to any pigment that gives colour to keratinous materials. Their solubility in water at 25° C. and at atmospheric pressure (760 mmHg) is less than 0.05% by weight, and preferably less than 0.01%.

The pigments that may be used are notably chosen from the organic and/or mineral pigments known in the art, notably those described in Kirk-Othmer's Encyclopedia of Chemical Technology and in Ullmann's Encyclopedia of Industrial Chemistry.

They may be natural, of natural origin, or non-natural.

These pigments may be in pigment powder or paste form. They may be coated or uncoated.

The pigments may be chosen, for example, from mineral pigments, organic pigments, lakes, pigments with special effects such as nacres or glitter flakes, and mixtures thereof.

The pigment may be a mineral pigment. The term “mineral pigment” refers to any pigment that satisfies the definition in Ullmann's encyclopaedia in the chapter on inorganic pigments. Mention may be made, among the inorganic pigments of use in the present invention, of iron oxides, chromium oxides, manganese violet, ultramarine blue, chromium hydrate, ferric blue and titanium oxide.

The pigment may be an organic pigment. The term “organic pigment” refers to any pigment that satisfies the definition in Ullmann's encyclopaedia in the chapter on organic pigments.

The organic pigment may notably be chosen from nitroso, nitro, azo, xanthene, pyrene, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds.

In particular, the white or coloured organic pigments may be chosen from carmine, carbon black, aniline black, azo yellow, quinacridone, phthalocyanine blue, the blue pigments codified in the Color Index under the references CI 42090, 69800, 69825, 74100, 74160, the yellow pigments codified in the Color Index under the references CI 11680, 11710, 19140, 20040, 21100, 21108, 47000, 47005, the green pigments codified in the Color Index under the references CI 61565, 61570, 74260, the orange pigments codified in the Color Index under the references CI 11725, 45370, 71105, the red pigments codified in the Color Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names:
Cosmenyl Yellow 10G: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may be composed notably of particles including an inorganic core, at least one binder for attaching the organic pigments to the core, and at least one organic pigment which at least partially covers the core.

The organic pigment may also be a lake. The term “lake” refers to dyes adsorbed onto insoluble particles, the assembly thus obtained remaining insoluble during use.

The mineral substrates onto which the dyes are adsorbed are, for example, alumina, silica, calcium sodium borosilicate or calcium aluminium borosilicate and aluminium.

Among the dyes, mention may be made of carminic acid. Mention may also be made of the dyes known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of pigments with special effects exist: those with a low refractive index, such as fluorescent or photochromic pigments, and those with a higher refractive index, such as nacres, interference pigments or glitter flakes.

Examples of pigments with special effects that may be mentioned include nacreous pigments such as mica covered with titanium or with bismuth oxychloride, coloured nacreous pigments such as mica covered with titanium and with iron oxides, mica covered with iron oxide, mica covered with titanium and notably with ferric blue or with chromium oxide, mica covered with titanium and with an organic pigment as defined previously, and also nacreous pigments based on bismuth oxychloride. Nacreous pigments that may be mentioned include the nacres Cellini sold by BASF (mica-TiO₂-lake), Prestige sold by Eckart (mica-TiO₂), Prestige Bronze sold by Eckart (mica-Fe₂O₃) and Colorona sold by Merck (mica-TiO₂—Fe₂O₃).

Mention may also be made of the gold-coloured nacres sold notably by the company BASF under the name Brilliant gold 212G (Timica), Gold 222C (Cloisonne), Sparkle gold (Timica), Gold 4504 (Chromalite) and Monarch gold 233X (Cloisonne); the bronze nacres sold notably by the company Merck under the name Bronze fine (17384) (Colorona) and Bronze (17353) (Colorona) and by the company BASF under the name Super bronze (Cloisonne); the orange nacres sold notably by the company BASF under the name Orange 363C (Cloisonne) and Orange MCR 101 (Cosmica) and by the company Merck under the name Passion orange (Colorona) and Matte orange (17449) (Microna); the brown nacres sold notably by the company BASF under the name Nu-antique copper 340XB (Cloisonne) and Brown CL4509 (Chromalite); the nacres with a copper tint sold notably by the company BASF under the name Copper 340A (Timica); the nacres with a red tint sold notably by the company Merck under the name Sienna fine (17386) (Colorona); the nacres with a yellow tint sold notably by the company BASF under the name Yellow (4502) (Chromalite); the red nacres with a gold tint sold notably by the company BASF under the name Sunstone G012 (Gemtone); the pink nacres sold notably by the company BASF under the name Tan opale G005 (Gemtone); the black nacres with a gold tint sold notably by the company BASF under the name Nu antique bronze 240 AB (Timica), the blue nacres sold notably by the company Merck under the name Matte blue (17433) (Microna), the white nacres with a silvery tint sold notably by the company Merck under the name Xirona Silver, and the golden-green pink-orange nacres sold notably by the company Merck under the name Indian summer (Xirona), and mixtures thereof.

Still as examples of nacreous agents, mention may also be made of particles including a borosilicate substrate coated with titanium oxide.

Particles comprising a glass substrate coated with titanium oxide are notably sold under the name Metashine MC108ORY by the company Toyal.

Finally, examples of nacres that may also be mentioned include polyethylene terephthalate glitter flakes, notably those sold by the company Meadowbrook Inventions under the name Silver 1P 0.004×0.004 (silver glitter flakes). It is also possible to envisage multilayer pigments based on synthetic substrates, such as alumina, silica, calcium sodium borosilicate, calcium aluminium borosilicate and aluminium.

The pigments with special effects may also be chosen from reflective particles, i.e. notably from particles whose size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. brighter points that contrast with their environment, making them appear to sparkle.

The reflective particles may be selected so as not to significantly alter the colouring effect generated by the colouring agents with which they are combined, and more particularly so as to optimize this effect in terms of colour rendition. They may more particularly have a yellow, pink, red, bronze, orange, brown, gold and/or coppery colour or tint.

These particles may have varied forms and may notably be in platelet or globular form, in particular in spherical form.

The reflective particles, whatever their form, may or may not have a multilayer structure and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically.

When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or inorganic materials.

More particularly, it may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Reflective particles are notably described in documents JP-A-09188830, JP-A-10158450, JP-A-10158541, JP-A-07258460 and JP-A-05017710.

Again as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by the company Toyal.

Particles with a glass substrate coated with nickel/chromium/molybdenum alloy are sold under the names Crystal Star GF 550 and GF 2525 by this same company.

Use may also be made of particles comprising a metal substrate, such as silver, aluminium, iron, chromium, nickel, molybdenum, gold, copper, zinc, tin, magnesium, steel, bronze or titanium, said substrate being coated with at least one layer of at least one metal oxide, such as titanium oxide, aluminium oxide, iron oxide, cerium oxide, chromium oxide, silicon oxides and mixtures thereof.

Examples that may be mentioned include aluminium powder, bronze powder or copper powder coated with SiO₂ sold under the name Visionaire by the company Eckart.

Mention may also be made of pigments with an interference effect which are not attached to a substrate, such as liquid crystals (Helicones HC from Wacker) or interference holographic glitter flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colours, and also particular optical effects such as metallic effects or interference effects.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

The dispersant serves to protect the dispersed particles against their agglomeration or flocculation. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C8 to C20 fatty acid and of polyols such as glycerol or diglycerol are used, such as poly(12-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the composition may be surface-treated with an organic agent.

Thus, the pigments surface-treated beforehand that are useful in the context of the invention are pigments which have been completely or partially subjected to a surface treatment of chemical, electronic, electrochemical, mechanochemical or mechanical nature with an organic agent, such as those described notably in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof, anionic surfactants; lecithins; sodium, potassium, magnesium, iron, titanium, zinc or aluminium salts of fatty acids, for example aluminium stearate or laurate; metal alkoxides; polyethylene; (meth)acrylic polymers, for example polymethyl methacrylates; polymers and copolymers containing acrylate units; alkanolamines; silicone compounds, for example silicones, notably polydimethylsiloxanes; organofluorine compounds, for example perfluoroalkyl ethers; fluorosilicone compounds.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in patent U.S. Pat. No. 4,578,266.

An organic agent covalently bonded to the pigments will preferably be used.

The agent for the surface treatment may represent from 0.1% to 50% by weight relative to the total weight of the surface-treated pigment, preferably from 0.5% to 30% by weight and even more preferentially from 1% to 20% by weight relative to the total weight of the surface-treated pigment.

Preferably, the surface treatments of the pigments are chosen from the following treatments:
a PEG-silicone treatment, for instance the AQ surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a magnesium myristate treatment, for instance the MM surface treatment sold by LCW;
an aluminium dimyristate treatment, such as the MI surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrogenosiloxane/perfluoroalkyl phosphate treatment, for instance the FSO1 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.

According to a particular embodiment of the invention, the dispersant is present with organic or mineral pigments in submicron-sized particulate form in composition B.

The term “submicron” or “submicronic” refers to pigments having a particle size that has been micronized by a micronization method and having a mean particle size of less than a micrometre (μm), in particular between 0.1 and 0.9 μm, and preferably between 0.2 and 0.6 μm.

According to one embodiment, the dispersant and the pigment(s) are present in an amount (dispersant:pigment), according to a weight ratio, of between 1:4 and 4:1, particularly between 1.5:3.5 and 3.5:1 or better still between 1.75:3 and 3:1.

The dispersant(s) may therefore have a silicone backbone, such as silicone polyether and dispersants of amino silicone type different from the amino silicones mentioned previously. Among the suitable dispersants that may be mentioned are:
aminosilicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxyl groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of amino silicone type different from the amino silicones mentioned previously and are cationic.

Preferably, the pigment(s) are chosen from mineral, mixed mineral-organic or organic pigments.

In one variant of the invention, the pigment(s) are organic pigments, preferentially organic pigments surface-treated with an organic agent chosen from silicone compounds. In another variant of the invention, the pigment(s) are mineral pigments.

Preferably, the pigment(s) are chosen from iron oxides, notably red, brown or black iron oxides. By way of example of iron oxide, mention may be made of the iron oxide sold by the company Sun Chemical under the name SunPuro® Red Iron Oxide.

### Direct Dye

Composition C and/or composition D employed within the context of the process according to the invention may comprise one or more direct dyes.

The term “direct dye” means natural and/or synthetic dyes, other than oxidation dyes. These are dyes that will spread superficially on the fibre.

They may be ionic or nonionic, preferably cationic or nonionic.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (XIV) and (XV) and the azo cationic dyes (XVI) and (XVII) below:

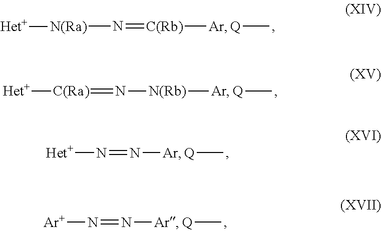

in which formulae (XIV) to (XVII):
Het⁺ represents a cationic heteroaryl radical, preferentially bearing an endocyclic cationic charge, such as imidazolium, indolium or pyridinium, which is optionally substituted, preferentially with at least one (C₁-C₈) alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group; or else the substituent Ra with a substituent of Het⁺ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q- represents an organic or mineral anionic counterion, such as a halide or an alkyl sulfate.

In particular, mention may be made of the azo and hydrazono direct dyes bearing an endocyclic cationic charge of formulae (XIV) to (XVII) as defined previously, more particularly, the cationic direct dyes bearing an endocyclic cationic charge described in patent applications WO 95/15144, WO 95/01772 and EP 714 954, preferentially the following direct dyes:

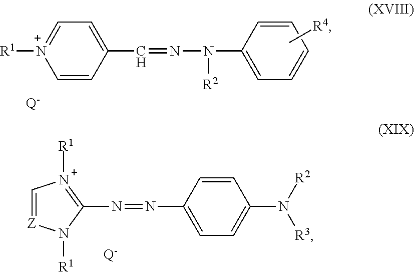

in which formulae (XVIII) and (XIX):
   R¹ represents a (C₁-C₄)alkyl group such as methyl;
   R² and R³, which may be identical or different, represent a hydrogen atom or a (C₁-C₄)alkyl group, such as methyl; and
   R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; in particular, R⁴ is a hydrogen atom;
   Z represents a CH group or a nitrogen atom, preferentially CH,
   Q- is an anionic counterion as defined previously, in particular a halide, such as chloride, or an alkyl sulfate, such as methyl sulfate or mesyl.

In particular, the dyes of formulae (XVIII) and (XIX) are chosen from Basic Red 51, Basic Yellow 87 and Basic Orange 31 or derivatives thereof with Q′ being an anionic counterion as defined previously, particularly a halide such as chloride, or an alkyl sulfate such as methyl sulfate or mesyl.

The direct dyes may be chosen from anionic direct dyes. The anionic direct dyes of the invention are dyes commonly referred to as “acid” direct dyes owing to their affinity for alkaline substances. The term “anionic direct dye” means any direct dye including in its structure at least one CO₂R or SO₃R substituent with R denoting a hydrogen atom or a cation originating from a metal or an amine, or an ammonium ion. The anionic dyes may be chosen from direct nitro acid dyes, azo acid dyes, azine acid dyes, triarylmethane acid dyes, indoamine acid dyes, anthraquinone acid dyes, indigoid dyes and natural acid dyes.

As acid dyes that are useful for the invention, mention may be made of the dyes of formulae (XX), (XX′), (XXI), (XXI′), (XXII), (XXII′), (XXIII), (XXIII′), (XXIV), (XXV), (XXVI) and (XXVII) below:
a) the diaryl anionic azo dyes of formula (XX) or (XX′):

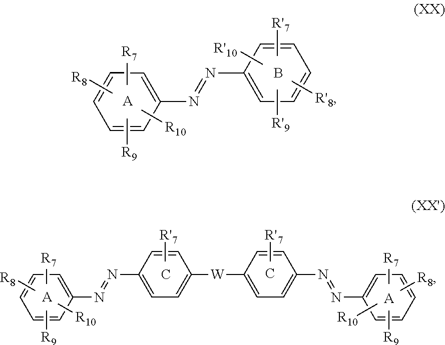

in which formulae (XX) and (XX′):
   R₇, R₈, R₉, R₁₀, R′₇, R′₈, R′₉ and R′₁₀, which may be identical or different, represent a hydrogen atom or a group chosen from:
   alkyl;
   alkoxy, alkylthio;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺ as defined previously;
   R″—S(O)₂—, with R″ representing a hydrogen atom or an alkyl, aryl, (di)(alkyl)amino or aryl(alkyl)amino group; preferentially a phenylamino or phenyl group;
   R′″—S(O)₂—X′— with R′″ representing an optionally substituted alkyl or aryl group, X′ as defined previously;
   (di)(alkyl)amino;
   aryl(alkyl)amino optionally substituted with one or more groups chosen from i) nitro; ii) nitroso; iii) (O)₂S(O⁻)—, M⁺ and iv) alkoxy with M⁺ as defined previously;
   optionally substituted heteroaryl; preferentially a benzothiazolyl group;
   cycloalkyl, notably cyclohexyl;
   Ar—N═N— with Ar representing an optionally substituted aryl group; preferentially a phenyl optionally substituted with one or more alkyl, (O)₂S(O⁻)—, M⁺or phenylamino groups;
   or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{o}—C(X)—X′—; viii) R^{o}—X′—C(X)—; ix) R^{o}—X′—C(X)—X″—; x) Ar—N═N— and xi) optionally substituted aryl(alkyl)amino; with M⁺, R^{o}, X, X′, X″ and Ar as defined previously;
   W represents a sigma bond a, an oxygen or sulfur atom, or a divalent radical i) —NR— with R as defined previously, or ii) methylene —C(Ra)(Rb)— with Ra and Rb, which may be identical or different, representing a hydrogen atom or an aryl group, or alternatively Ra and Rb form, together with the carbon atom that bears them, a spiro cycloalkyl; preferentially, W represents a sulfur atom or Ra and Rb together form a cyclohexyl;
it being understood that formulae (XX) and (XX′) comprise at least one sulfonate radical (O)₂S(O⁻)—, M⁺ or one carboxylate radical (O)CO⁻—, M⁺ on one of the rings A, A′, B, B′ or C; preferentially sodium sulfonate.

As examples of dyes of formula (XX), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3, Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Food Yellow 3 or Sunset Yellow;
and, as examples of dyes of formula (XX′), mention may be made of: Acid Red 111, Acid Red 134, Acid Yellow 38;
b) the pyrazolone anionic azo dyes of formulae (XXI) and (XXI′):

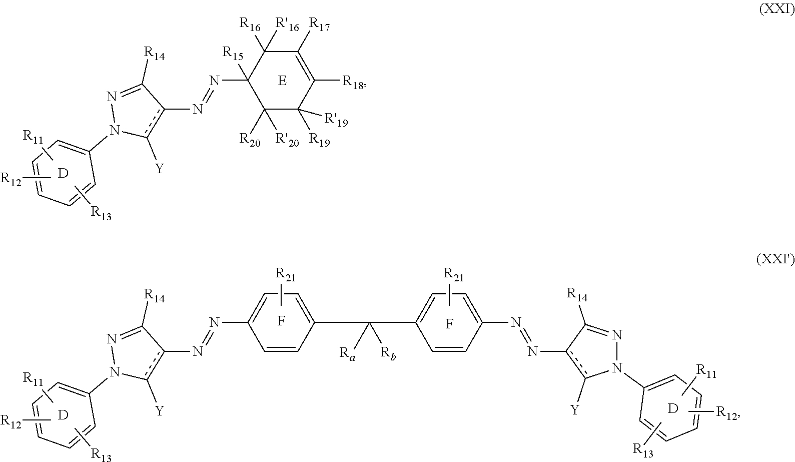

### (XXI′),

in which formulae (XXI) and (XXI′):
   R₁₁, R₁₂ and R₁₃, which may be identical or different, represent a hydrogen or halogen atom, an alkyl group or —(O)₂S(O⁻), M⁺ with M⁺ as defined previously;
   R₁₄ represents a hydrogen atom, an alkyl group or a group —C(O)O, M⁺ with M⁺ as defined previously;
   R₁₅ represents a hydrogen atom;
   R₁₆ represents an oxo group, in which case R′₁₆ is absent, or alternatively R₁₅ with R₁₆ together form a double bond;
   R₁₇ and R₁₈, which may be identical or different, represent a hydrogen atom, or a group chosen from:
   (O)₂S(O⁻)—, M⁺ with M⁺ as defined previously;
   Ar—O—S(O)₂— with Ar representing an optionally substituted aryl group; preferentially a phenyl optionally substituted with one or more alkyl groups;
   R₁₉ and R₂₀ together form either a double bond, or a benzo group D′, which is optionally substituted;
   R′₁₆, R′₁₉ and R′₂₀, which may be identical or different, represent a hydrogen atom or an alkyl or hydroxyl group;
   R₂₁ represents a hydrogen atom or an alkyl or alkoxy group;
   Ra and Rb, which may be identical or different, are as defined previously; preferentially, Ra represents a hydrogen atom and Rb represents an aryl group;
   Y represents either a hydroxyl group or an oxo group;
   [symbol] represents a single bond when Y is an oxo group; and represents a double bond when Y represents a hydroxyl group;
it being understood that formulae (XXI) and (XXI′) comprise at least one sulfonate radical (O)₂S(O⁻)—, M⁺ or one carboxylate radical —C(O)O, M⁺ on one of the rings D or E; preferentially sodium sulfonate.

As examples of dyes of formula (XXI), mention may be made of: Acid Red 195, Acid Yellow 23, Acid Yellow 27, Acid Yellow 76, and as examples of dyes of formula (XXI′), mention may be made of: Acid Yellow 17;
c) the anthraquinone dyes of formulae (XXII) and (XXII′):

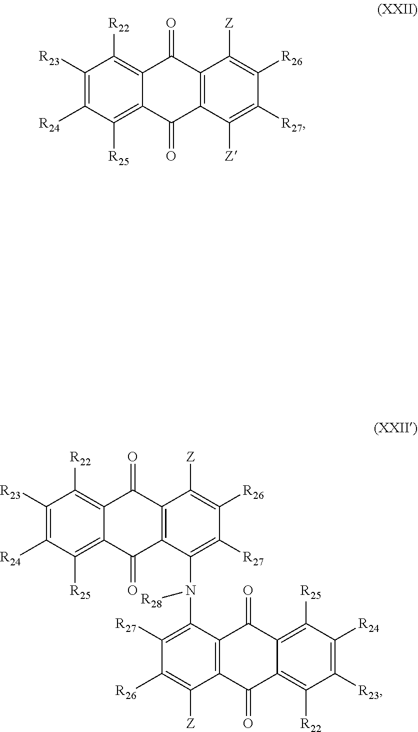

in which formulae (XXII) and (XXII′):
   R₂₂, R₂₃, R₂₄, R₂₅, R₂₆ and R₂₇, which may be identical or different, represent a hydrogen or halogen atom, or a group chosen from:
   alkyl;
   hydroxyl, mercapto;
   alkoxy, alkylthio;
   optionally substituted aryloxy or arylthio, preferentially substituted with one or more groups chosen from alkyl and (O)₂S(O⁻)—, M⁺ with M⁺ as defined previously;
   aryl(alkyl)amino optionally substituted with one or more groups chosen from alkyl and (O)₂S(O⁻)—, M⁺ with M⁺ as defined previously;
   (di)(alkyl)amino;
   (di)(hydroxyalkyl)amino;
   (O)₂S(O⁻)—, M⁺ with M⁺ as defined previously;
   Z′ represents a hydrogen atom or a group NR₂₈R₂₉ with R₂₈ and R₂₉, which may be identical or different, representing a hydrogen atom or a group chosen from:
   alkyl;
   polyhydroxyalkyl such as hydroxyethyl;
   aryl optionally substituted with one or more groups, particularly i) alkyl such as methyl, n-dodecyl, n-butyl; ii) (O)₂S(O⁻)—, M⁺ with M⁺ as defined previously; iii) R^{o}—C(X)—X′—, R^{o}—X′—C(X)—, R^{o}—X′—C(X)—X″— with R^{o}, X, X′ and X″ as defined previously, preferentially R^{o} represents an alkyl group;
   cycloalkyl; notably cyclohexyl;
   Z represents a group chosen from hydroxyl and NR′₂₈R′₂₉ with R′₂₈ and R′₂₉, which may be identical or different, representing the same atoms or groups as R₂₈ and R₂₉ as defined previously;
it being understood that formulae (XXII) and (XXII′) comprise at least one sulfonate radical (O)₂S(O⁻)—, M⁺ or one carboxylate radical C(O)O, M⁺; preferentially sodium sulfonate.

As examples of dyes of formula (XXII), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT Violet No. 2; and, as an example of a dye of formula (XXII′), mention may be made of: Acid Black 48.
d) the nitro dyes of formulae (XXIII) and (XXIII′):

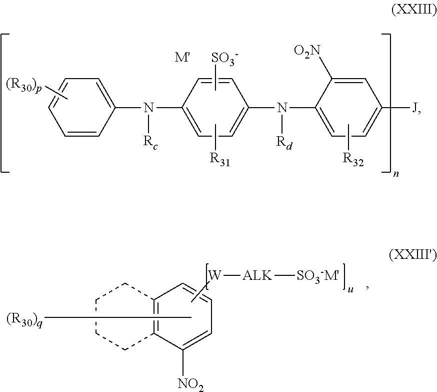

in which formulae (XXIII) and (XXIII′):
   R₃N, R₃₁ and R₃₂, which may be identical or different, represent a hydrogen or halogen atom, or a group chosen from:
   alkyl;
   alkoxy optionally substituted with one or more hydroxyl groups, alkylthio optionally substituted with one or more hydroxyl groups;
   hydroxyl, mercapto;
   nitro, nitroso;
   polyhaloalkyl;
   R^{o}—C(X)—X′—, R^{o}—X′—C(X)—, R^{o}—X′—C(X)—X″— with R^{o}, X, X′ and X″ as defined previously;
   (O)₂S(O⁻)—, M⁺ with M⁺ as defined previously;
   (O)CO⁻—, M⁺ with M⁺as defined previously;
   (di)(alkyl)amino;
   (di)(hydroxyalkyl)amino;
   heterocycloalkyl such as piperidino, piperazino or morpholino; in particular, R₃₀, R₃₁ and R₃₂ represent a hydrogen atom;
   Rc and Rd, which may be identical or different, represent a hydrogen atom or an alkyl group;
   W is as defined previously; W particularly represents an —NH— group;
   ALK represents a linear or branched divalent C₁-C₆ alkylene group; in particular, ALK represents a —CH₂—CH₂— group;
   n is 1 or 2;
   p represents an integer inclusively between 1 and 5;
   q represents an integer inclusively between 1 and 4;
   u is 0 or 1;
   when n is 1, J represents a nitro or nitroso group; particularly nitro;
   when n is 2, J represents an oxygen or sulfur atom, or a divalent radical —S(O)ₘ— with m representing an integer 1 or 2; preferentially, J represents an —SO₂— radical;
   M′ represents a hydrogen atom or a cationic counterion;

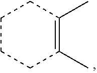

which may be present or absent, represents a benzo group optionally substituted with one or more groups R₃o as defined previously;
it being understood that formulae (XXIII) and (XXIII′) comprise at least one sulfonate radical (O)₂S(O⁻)—, M⁺or one carboxylate radical C(O)O, M⁺; preferentially sodium sulfonate.

As examples of dyes of formula (XXIII), mention may be made of: Acid Brown 13 and Acid Orange 3; as examples of dyes of formula (XXIII′), mention may be made of: Acid Yellow 1, the sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-p-hydroxyethylamino-3-nitrobenzenesulfonic acid; EXT D&C Yellow 7;
e) the triarylmethane dyes of formula (XXIV):

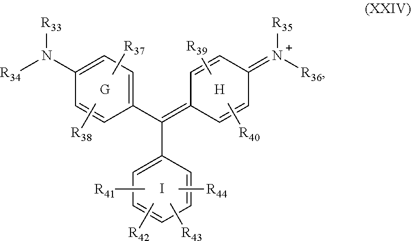

in which formula (XXIV):
   R₃₃, R₃₄, R₃₅ and R₃₆, which may be identical or different, represent a hydrogen atom or a group chosen from alkyl, optionally substituted aryl and optionally substituted arylalkyl; particularly an alkyl and benzyl group optionally substituted with a group (O)ₘS(O⁻)—, M⁺ with M⁺ and m as defined previously;
   R₃₇, R₃₈, R₃₉, R₄o, R₄₁, R₄₂, R₄₃ and R₄₄, which may be identical or different, represent a hydrogen atom or a group chosen from:
   alkyl;
   alkoxy, alkylthio;
   (di)(alkyl)amino;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺as defined previously;
   or alternatively two contiguous groups R₄₁ with R₄₂ or R₄₂ with R₄₃ or R₄₃ with R₄₄ together form a fused benzo group: I′; with I′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{o}—C(X)—X′—; viii) R^{o}—X′—C(X)— and ix) R^{o}—X′—C(X)—X″—; with M⁺, R^{o}, X, X′ and X″ as defined previously;
in particular, R₃₇ to R₄o represent a hydrogen atom, and R₄₁ to R₄₄, which may be identical or different, represent a hydroxyl group or (O)₂S(O⁻)—, M⁺; and when R₄₃ with R₄₄ together form a benzo group, it is preferentially substituted with an (O)₂S(O⁻)— group;
it being understood that at least one of the rings G, H, I or I′ comprises at least one sulfonate radical (O)₂S(O⁻)— or one carboxylate radical —C(O)O; preferentially sulfonate.

As examples of dyes of formula (XXIV), mention may be made of: Acid Blue 1; Acid Blue 3; Acid Blue 7, Acid Blue 9; Acid Violet 49; Acid Green 3; Acid Green 5 and Acid Green 50.
f) the xanthene-based dyes of formula (XXV):

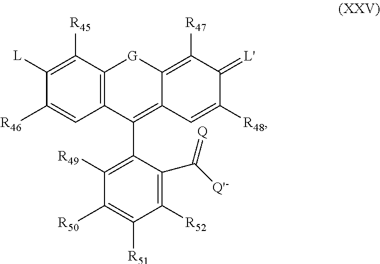

in which formula (XXV):
   R₄₅, R₄₆, R₄₇ and R₄₈, which may be identical or different, represent a hydrogen or halogen atom;
   R₄₉, R₅o, R₅₁ and R₅₂, which may be identical or different, represent a hydrogen or halogen atom, or a group chosen from:
   alkyl;
   alkoxy, alkylthio;
   hydroxyl, mercapto;
   nitro, nitroso;
   (O)₂S(O⁻)—, M⁺ with M⁺ representing a hydrogen atom or a cationic counterion;
   (O)CO⁻—, M⁺ with M⁺ as defined previously;
particularly, R₄₉, R₅₀, R₅₁ and R₅₂ represent a hydrogen or halogen atom;
   G represents an oxygen or sulfur atom or a group NRe with Re as defined previously; particularly, G represents an oxygen atom;
   L represents an alkoxide O⁻, M⁺; a thioalkoxide S⁻, M⁺ or a group NRf, with Rf representing a hydrogen atom or an alkyl group, and M⁺ as defined previously; M⁺ is particularly sodium or potassium;
   L′ represents an oxygen or sulfur atom or an ammonium group: N⁺RfRg, with Rf and Rg, which may be identical or different, representing a hydrogen atom or an optionally substituted alkyl or aryl group; L′ particularly represents an oxygen atom or a phenylamino group optionally substituted with one or more alkyl or (O)ₘS(O⁻)—, M⁺ groups with m and M⁺ as defined previously;
   Q and Q′, which may be identical or different, represent an oxygen or sulfur atom; particularly, Q and Q′ represent an oxygen atom;
   M⁺ is as defined previously.

As examples of dyes of formula (XXV), mention may be made of: Acid Yellow 73; Acid Red 51; Acid Red 52; Acid Red 87; Acid Red 92; Acid Red 95; Acid Violet 9.
g) the indole-based dyes of formula (XXVI):

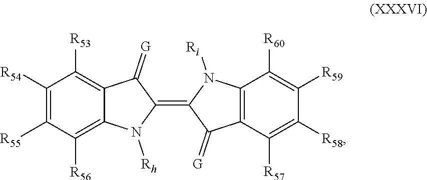

in which formula (XXVI):
   R₅₃, R₅₄, R₅₅, R₅₆, R₅₇, R₅₈, R₅₉ and R₆o, which may be identical or different, represent a hydrogen atom or a group chosen from:
   alkyl;
   alkoxy, alkylthio;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺as defined previously;
   G represents an oxygen or sulfur atom or a group NRe with Re as defined previously; particularly, G represents an oxygen atom;
   Ri and Rh, which may be identical or different, represent a hydrogen atom or an alkyl group;
it being understood that formula (XXVI) comprises at least one sulfonate radical (O)₂S(O⁻)—, M⁺ or one carboxylate radical —C(O)O, M⁺; preferentially sodium sulfonate.

As examples of dyes of formula (XXVI), mention may be made of: Acid Blue 74;
h) the quinoline-based dyes of formula (XXVII):

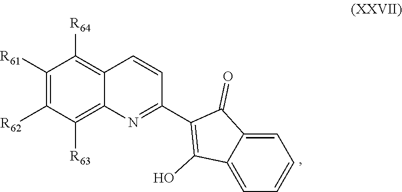

in which formula (XXVII):
   R₆₁ represents a hydrogen or halogen atom or an alkyl group;
   R₆₂, R₆₃ and R₆₄, which may be identical or different, represent a hydrogen atom or a group (O)₂S(O⁻)—, M⁺ with M⁺ representing a hydrogen atom or a cationic counterion;
or alternatively R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;
it being understood that formula (XXVII) comprises at least one sulfonate radical (O)₂S(O⁻)—, M⁺, preferentially sodium sulfonate.

As examples of dyes of formula (XXVII), mention may be made of: Acid Yellow 2, Acid Yellow 3 and Acid Yellow 5.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

Preferably, the direct dyes are chosen from anionic direct dyes.

The colouring agent(s) may be present in a total amount ranging from 0.001% to 20% by weight and preferably from 0.005% to 15% by weight relative to the total weight of composition C and/or of composition D; preferably, the colouring agents are chosen from pigments.

The pigment(s) may be present in a total amount ranging from 0.05% to 20% by weight, preferably from 0.1% to 15% by weight and better still from 0.5% to 12% by weight, relative to the total weight of composition C and/or of composition D.

The direct dye(s) may be present in a total amount ranging from 0.001% to 10% by weight relative to the total weight of the composition, preferably from 0.005% to 5% by weight relative to the total weight of composition C and/or of composition D.

Preferably, composition D comprises at least one colouring agent chosen from pigments, direct dyes and mixtures thereof.

Preferably, composition C does not comprise at least one colouring agent chosen from pigments, direct dyes and mixtures thereof.

### Additives

Composition C and/or composition D employed within the context of the process according to the invention may contain any adjuvant or additive usually used.

Among the additives that may be contained in composition C, mention may be made of reducing agents, softeners, antifoams, moisturizers, UV-screening agents, peptizers, solubilizers, fragrances, anionic, cationic, nonionic or amphoteric surfactants, proteins, vitamins, polymers other than the polymers described previously, thickeners other than the polymers described previously, preserving agents, oils, waxes and mixtures thereof.

Composition C and/or composition D employed within the context of the process according to the invention may notably be in the form of a suspension, a dispersion, a gel, an emulsion, notably an oil-in-water (O/W) or water-in-oil (W/O) emulsion, or a multiple emulsion (W/O/W or polyol/O/W or O/W/O), in the form of a cream, a mousse, a stick, a dispersion of vesicles, notably of ionic or nonionic lipids, or a two-phase or multi-phase lotion.

A person skilled in the art may select the appropriate presentation form, and also the method for preparing it, on the basis of his general knowledge, taking into account firstly the nature of the constituents used, notably their solubility in the support, and secondly the intended application of the composition.

### Protocol

Composition C and composition D described above may be used on wet or dry hair, and also on any type of fair or dark, natural or coloured, permanent-waved, bleached or relaxed hair.

According to a preferred embodiment, composition C and composition D are applied simultaneously to the keratinous fibres.

According to another preferred embodiment, composition D is applied to the hair after applying composition C to the hair.

According to another preferred embodiment, composition D is applied to the hair before applying composition C to the hair.

More preferentially, composition D is applied to the hair after applying composition C to the hair.

According to a particular embodiment of the invention, the hair is washed before applying composition C and composition D.

Preferably, a washing, rinsing, draining or drying step is performed after applying composition C to the hair and before applying composition D to the hair.

More preferentially, a drying step is performed after applying composition C to the hair and before applying composition D to the hair, preferably, the drying step is carried out with the aid of a hairdryer, for example at a temperature of greater than or equal to 30° C.

The application to the hair may be performed via any standard means, in particular using a comb, a fine brush, a coarse brush, a sponge or with the fingers.

The application of composition C and of composition D to the hair is generally performed at room temperature (between 15 and 25° C.).

After applying composition C to the hair, it is possible to wait for between 1 minute and 6 hours, in particular between 1 minute and 2 hours, more particularly between 1 minute and 1 hour, more preferentially between 1 minute and 30 minutes, before, for example, applying composition D to the hair or, for example, a washing, rinsing, draining or drying step.

Preferably, there is no leave-on time after applying composition C to the hair and before applying composition D to the hair.

After applying composition C and composition D, the hair may be left to dry or may be dried, for example at a temperature of greater than or equal to 30° C.

The process according to the invention may thus comprise a step of applying heat to the keratinous fibres using a heating tool.

The heat application step of the process of the invention may be performed using a hood, a hairdryer, a straightening iron, a curling iron, a Climazon, etc.

Preferably, the heat application step of the process of the invention is performed using a hairdryer.

When the process of the invention involves a step of applying heat to the hair, the step of applying heat to the hair takes place after applying composition C and composition D to the hair.

During the step of applying heat to the hair, a mechanical action may be exerted on the locks, such as combing, brushing or running the fingers through.

When the step of applying heat to the hair is performed using a hood or a hairdryer, the temperature is preferably between 30° C. and 110° C., preferentially between 50° C. and 90° C.

When the step of applying heat to the hair is performed using a straightening iron, the temperature is preferably between 110° C. and 220° C., preferably between 140° C. and 200° C.

In a particular variant, the process of the invention involves a step (c1) of applying heat using a hood, a hairdryer or a Climazon, preferably a hairdryer, and a step (c2) of applying heat using a straightening or curling iron, preferably a straightening iron.

Step (c1) may be performed before step (c2).

During step (c1), also referred to as the drying step, the hair may be dried, for example at a temperature above or equal to 30° C. According to a particular embodiment, this temperature is above 40° C. According to a particular embodiment, this temperature is above 45° C. and below 110° C.

Preferably, if the hair is dried, it is dried, in addition to a supply of heat, with a flow of air. This flow of air during drying makes it possible to improve the strand separation of the coating.

During drying, a mechanical action may be exerted on the locks, such as combing, brushing or running the fingers through.

During step (c2), the passage of the straightening or curling iron, preferably the straightening iron, may be performed at a temperature ranging from 110° C. to 220° C., preferably between 140° C. and 200° C.

After the drying step, a shaping step may be performed, for example with a straightening iron; the temperature for the shaping step is between 11° and 220° C., preferably between 14° and 200° C.

Preferably, the invention is a process for colouring the hair comprising the following steps:
a) the application to the hair of at least one composition C comprising:
   one or more (poly)carbodiimide compound(s) as described previously, and then
ii) optionally a leave-on time of said composition C on the hair of from 1 minute to 30 minutes, preferably from 1 to 20 minutes; and then
iii) optionally a step of washing, rinsing, draining or drying said hair, and then
b) the application to the hair of at least one composition D comprising at least one silicone elastomer containing carboxylic acid functions as described previously; and then
v) optionally a leave-on time of said composition D on the hair of from 1 minute to 30 minutes, preferably from 1 to 20 minutes; and then
vi) optionally a step of washing, rinsing, draining or drying the hair.

Preferably, the step of applying composition C and/or composition D to the hair is repeated several times.

According to a preferred embodiment, the colouring process according to the invention is a process for colouring the hair which consists in extemporaneously mixing, at the time of use, at least two compositions C and D to obtain a composition E and in applying composition E to the hair, with:
composition C comprising one or more (poly)carbodiimide compound(s) as described previously;
composition D comprising at least one silicone elastomer containing carboxylic acid functions as described previously,
composition C and/or composition D comprising at least one colouring agent chosen from pigments, direct dyes and mixtures thereof.

Preferably, composition D comprises at least one colouring agent chosen from pigments, direct dyes and mixtures thereof.

Preferably, composition C does not comprise at least one colouring agent chosen from pigments, direct dyes and mixtures thereof.

According to this embodiment, compositions C and D are mixed preferably less than 15 minutes before application to the hair, more preferentially less than 10 minutes before application, better still less than 5 minutes before application.

The weight ratio between composition C and composition D preferably ranges from 0.1 to 10, preferentially from 0.2 to 5 and better still from 0.5 to 2, or even from 0.6 to 1.5. In a particular embodiment, the weight ratio between composition C and composition D is equal to 1.

### Makeup-Removing Composition:

The process according to the invention may comprise a step of applying a makeup-removing composition to coloured keratin fibres such as the hair.

Preferably, the process according to the invention comprises a step of applying a makeup-removing composition to the keratin fibres after the application of composition C and composition D.

The makeup-removing composition may correspond to a makeup-removing composition conventionally used in cosmetics.

Preferably, the makeup-removing composition comprises at least one compound chosen from surfactants, solvents, fatty substances, acids, bases and mixtures thereof.

According to a preferred embodiment, the makeup-removing composition comprises at least one hydrocarbon-based oil.

Preferably, the hydrocarbon-based oil or oils are chosen from synthetic esters of formula R1COOR2 in which R1 represents the residue of a fatty acid including from 8 to 29 carbon atoms, and R2 represents a branched or unbranched hydrocarbon-based chain containing from 3 to 30 carbon atoms, and mixtures thereof, more preferentially from isopropyl myristate, isononyl isononanoate and/or mixtures thereof.

The application of composition C and composition D to keratin fibres such as the hair is performed before the application of a makeup-removing composition.

The step of applying heat to the keratin fibres may be performed after the application of composition C and composition D and before the application of the makeup-removing composition to the keratin fibres.

The application of the makeup-removing composition may be performed on dry coloured keratin fibres or wet coloured keratin fibres and also on all types of fibres.

The makeup removal process is generally performed at room temperature (between 15 and 25° C.).

The application of the makeup-removing composition may be performed immediately after the application of composition C and composition D (i.e. a few minutes to a few hours after the application of the colouring composition), or in the days or weeks following the application of the colouring composition.

The makeup-removing composition may be applied using any suitable medium, notably one that is capable of absorbing it, for example a fibrous makeup-removing disc, e.g. a woven or non-woven, wadding, flocked film, sponge, wipe, or a twisted or injection-moulded mascara application brush.

The makeup-removing composition may be contained in a container and removed as required for each makeup removal operation. As a variant, the makeup-removing composition impregnates the support used for the makeup removal, in which case the carrier may be packaged, for example, in leaktight packaging.

After using the makeup-removing composition, the keratin fibres may be left unrinsed. Alternatively, they may be rinsed. Rinsing may be performed, for example, with running water, without the addition of soap.

### Multi-Compartment Device (Kit)

The present invention also relates to a device for colouring the hair comprising at least two compartments containing:
in a first compartment, a composition C comprising:
one or more (poly)carbodiimide compounds as described previously; and
in a second compartment, a composition D comprising at least one silicone elastomer containing carboxylic acid functions as described previously;
composition C and/or composition D comprising at least one colouring agent chosen from pigments, direct dyes and mixtures thereof.

According to a preferred variant of the invention, the device for colouring the hair comprises at least two compartments containing:
in a first compartment, a composition C as described previously;
in a second compartment, a composition D as described previously; and
optionally, in a third compartment, a makeup-removing composition as described previously.

The present invention will now be described more specifically by means of examples, which are in no way limiting of the scope of the invention. However, the examples make it possible to support specific features, variants and preferred embodiments of the invention.

### EXAMPLES

The (poly)carbodiimide(s) of the invention are accessible via synthetic methods known to those skilled in the art starting from commercial products or reagents that can be synthesized according to chemical reactions that are also known to those skilled in the art. Mention may be made, for example, of the book Sciences of Synthesis—Houben—Weyl Methods of Molecular Transformations, 2005, Georg Thiem Verlag Kg, Rudigerstrasse 14, D-70469 Stuttgart, or the American patent U.S. Pat. No. 4,284,730 or the Canadian patent application CA 2 509 861.

More particularly, the process for preparing the (poly)carbodiimides of the invention involves, in a first step, a diisocyanate reagent (1):

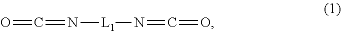

in which formula (1) L₁ is as defined previously, which reacts in the presence of a carboimidation catalyst (2) such as those described in U.S. Pat. No. 4,284,730, notably phosphorus-based catalysts particularly chosen from phospholene oxides and phospholene sulfoxides, diaza- and oxaza-phospholanes, preferably under an inert atmosphere (nitrogen or argon), and in particular in a polar solvent which is preferably aprotic such as THF, glyme, diglyme, 1,4-dioxane or DMF, at a temperature between room temperature and the reflux temperature of the solvent, preferably at about 140° C.; to give the carbodiimide diisocyanate compound (3):

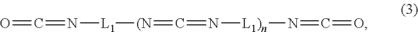

in which formula (3) L₁ and n are as defined previously. Benzoyl halogen such as benzoyl chloride may be added to deactivate the catalyst.

To obtain “symmetrical” (poly)carbodiimides, during the second step of the preparation process, compound (3) reacts with 1 molar equivalent (1 eq.) of nucleophilic reagent R₁—X₁—H and then 0.5 eq. of reagent H-E-H with R₁, X₁ and E as defined previously, to give the “symmetrical” compound (4) according to the invention:

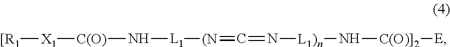

in which formula (4) R₁, X₁, L₁, n and E are as defined previously. According to one variant to obtain compound (4) from (3), it is possible first to add 0.5 eq. of reagent H-E-H and then 1 eq. of reagent R₁—X₁—H.

To obtain “dissymmetrical” (poly)carbodiimides, during the second step of the preparation process, compound (3) reacts with 1 molar equivalent (1 eq.) of nucleophilic reagent R₁—X₁—H and then 1 eq. of reagent H-E-H with R₁, X₁ and E as defined previously, to give compound (5):

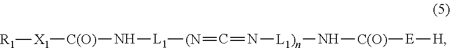

in which formula (5) R₁, X₁, L₁, n and E are as defined previously.

According to one variant to obtain compound (5) from (3), it is possible first to add 1 eq. of reagent R₁—X₁—H and then 0.5 eq. of reagent H-E-H.

During a third step, compound (5) reacts with 1 eq. of compound (6)

R₂—X₂—C(O)—NH-L₁-(N═C═N-L₁)_{z}-N═C═O (6),
said compound (6) is prepared beforehand from compound (3′)

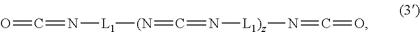

in which formula (3′) L₁ and z are as defined previously, which reacts with 1 eq. of nucleophilic reagent R₂—X₂—H with L₁, R₂, X₂ and z as defined previously, to give the dissymmetrical compound (7):

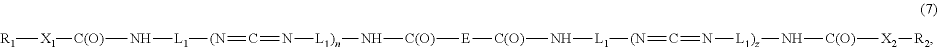

in which formula (7) R₁, X₁, L₁, R₂, X₂, n, z and E are as defined previously.

It is also possible to react 1 molar equivalent of compound O═C═N-L₁-(N═C═N-L₁)_{z}-N═C═O (3′) with 1/w molar equivalent of H-E-H, followed by 1 eq. of nucleophilic reagent R₂—X₂—H to give compound (8):

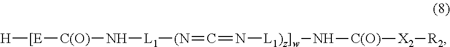

in which formula (8) L₁, R₂, X₂, z and E are as defined previously, and w is an integer between 1 and 3; more preferentially, w=1.

This last compound (8) can then react with 1 eq. of compound (4′):

R₁—X₁—C(O)—NH-L₁-(N═C═N-L₁)ₙ-N═C═O (4′),
(said compound (4′) being able to be synthesized by reaction of 0.5 eq. of nucleophilic reagent R₁—X₁—H with 1 equivalent of compound (3)), to give the (poly)carbodiimide (9) of the invention:

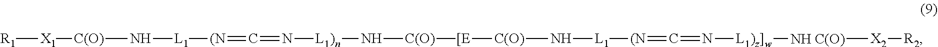

in which formula (9) L₁, R₁, X₁, R₂, X₂, n, z, w and E are as defined previously.

The (poly)carbodiimide compounds, and similarly all the reaction intermediates and reagents, may be purified via conventional methods known to those skilled in the art, such as extraction with water and water-immiscible organic solvent, precipitation, centrifugation, filtration and/or chromatography.

### Example 1: Process for Synthesizing the (Poly)Carbodiimide Compound

50 g of 4,4′-dicyclohexylmethane diisocyanate and 0.5 g of 4,5-dihydro-3-methyl-1-phenyl-1H-phosphole 1-oxide were placed with stirring in a 500 mL three-necked round-bottomed flask equipped with a thermometer, a stirrer and a reflux tube.

The reaction medium was heated at 140° C. under nitrogen for 4 hours, the reaction being monitored by infrared spectroscopy by means of the absorption of the isocyanate functions between 2200 and 2300 cm⁻¹, and then cooled to 120° C.

A mixture of 5.3 g of polyethylene glycol monomethyl ether and 1.2 g of 1,4-butanediol are introduced with stirring into the reaction medium. The temperature of 120° C. is maintained until the isocyanate functions have totally disappeared, monitored by infrared spectroscopy at 2200-2300 cm⁻¹, and is then cooled to room temperature.

After cooling to room temperature, the reaction medium is poured dropwise with vigorous stirring into a 500 mL glass beaker containing 85 g of distilled water, to give the desired product in the form of a translucent yellow liquid.

### Example 2

Composition C as described below was prepared: the amounts are expressed as g of starting material as obtained/100 g, unless otherwise mentioned. In the table 1 below, “a.m.” means “active material”.

TABLE 1

| Composition | C |
|---|---|
| Polycarbodiimide⁽¹⁾ | 24 (9.6 a.m). |
| Hydroxyethyl acrylate/sodium | 1 (0.9 a.m). |
| acryloyldimethyl taurate copolymer ⁽²⁾ | |
| Amodimethicone (and) Trideceth-5 (and) | 5 (0.75 a.m). |
| Trideceth-10 ⁽³⁾ | |
| Preserving agent(s) | qs |
| Water | qs 100 |

(1) synthesized according to the synthetic process described in example 1 (containing 40% active material in water),
(2) sold by the company SEPPIC under the name Sepinov EMT10 (containing 90% active material),
(3) sold by the company Wacker under the name Belsil ADM LOG 1 (containing 15% active material).

Compositions D1 and D2 as described below were prepared: the amounts are expressed as g of starting material as obtained/100 g.

TABLE 2

| Compositions | D1 | D2 |
|---|---|---|
| BIS-CARBOXYDECYL DIMETHICONE | — | 15.12 |
| (silicone elastomer containing carboxylic acid ) ⁽⁴⁾ | | |
| Isododecane (and) Hexyl/Succinyl dimethicone | 63 18.9 | |
| crosspolymer⁽⁵⁾ | a.m. | |
| Nylon-611/Dimethicone copolymer⁽⁶⁾ | 2.5 | — |
| Isopropyl alcohol | 3 | — |
| Red mineral pigment | 10 | 10 |
| Isododecane | qs 100 | qs 100 |

(4) sold by the company Momentive Performance Materials under the name Silform INX,
(5) sold by the company Dow Corning under the name DOWSIL EL-7314 SILICONE ELASTOMER BLEND® (containing 30% active material in the isododecane),
(6) sold by the company Dow Corning under the name DOWSIL 2-8179 GELLANT.

Composition C is applied to locks of natural hair containing 90% white hairs, in a proportion of 0.4 g of composition per gram of lock. The locks of hair are then combed and dried with a hairdryer.

Composition D1 or composition D2 is then applied to the locks of hair treated beforehand with composition C, in a proportion of 0.4 g of composition per gram of lock.

The locks of hair are then combed and dried with a hairdryer.

In another comparative process, only composition D is applied to locks of natural hair containing 90% white hairs, in a proportion of 0.4 g of composition per gram of lock. The locks of hair are then combed and dried with a hairdryer.

The locks of hair are then left at room temperature and humidity for 24 hours.

The different processes are summarized in the table below:

TABLE 3

| | 1 | 2 | 3 |
|---|---|---|---|
| Process | invention | (comparative) | (comparative) |
| Application of | Yes | Yes | No |
| composition C | | | |
| Application of | D1 | D2 | D1 |
| composition D | | | |

Thus, the locks of hair treated with compositions C then D1 are treated using process 1 according to the invention.

The locks of hair treated with compositions C then D2 or just D1 are treated using comparative processes 2 and 3.

The locks of hair thus coloured are then subjected to a test of several repeated shampoo washes so as to evaluate the fastness (persistence) of the colouring obtained with respect to shampoo washes, according to the shampoo washing protocol described below.

### Shampoo Washing Protocol:

The coloured locks of hair are combed, moistened with water at 35° C. and then passed between the fingers five times for 5 seconds. The locks of hair are then squeezed dry between two fingers.

A standard shampoo (Gamier Ultra Doux) is applied uniformly to the coloured locks, in a proportion of 0.4 g of standard shampoo per gram of locks, the locks of hair being massaged gently along the length (6 passes) for 15 seconds, from the root to the end.

The locks of hair are then placed on a watch glass and left to stand for 1 minute.

Next, the locks of hair are rinsed with water while passing the lock between the fingers (15 passes). The locks of hair are then squeezed dry between two fingers before the next shampoo wash.

Once the tests of several shampoo washes have been performed, the locks of hair are combed and dried with a hairdryer.

### Results

The persistence of the colour of the locks was evaluated in the CIE L* a* b* system, using a Minolta Spectrophotometer CM3600A colorimeter (illuminant D65, angle 10°, specular component included).

In this L*a*b* system, L* represents the intensity of the colour, a* indicates the green/red colour axis and b* the blue/yellow colour axis.

The persistence of the colouring is evaluated by the colour difference ΔE between the coloured locks before shampooing, then after having undergone five shampoo washes according to the protocol described above. The lower the ΔE value, the more persistent the colour with respect to shampoo washing.

The ΔE value is calculated according to the following equation:

$$Δ E = \sqrt{{( {L}^{*} - {{L}_{0}}^{*} )}^{2} + {( {a}^{*} - {{a}_{0}}^{*} )}^{2} + {( {b}^{*} - {{b}_{0}}^{*} )}^{2}}$$

In this equation, L*a*b* represent the values measured after colouring the hair and after performing the shampoo washes, and L₀*a₀*b₀* represent the values measured after colouring the hair but before shampoo washing.

TABLE 4

| | Number of | | | | |
|---|---|---|---|---|---|
| | shampoo | | | | |
| Processes | washes | L* | a* | b* | ΔE |
| 1 (Invention) | 0 | 37.2 | 25.9 | 19.8 | — |
| | 5 | 58.9 | 12.7 | 18.7 | 25.4 |
| 2 (comparative) | 0 | 34.4 | 27.5 | 21.9 | — |
| | 5 | 61.2 | 5.8 | 16.1 | 35.0 |
| 3 (comparative) | 0 | 36.1 | 24.6 | 18.7 | — |
| | 5 | 63.4 | 7.3 | 18.2 | 32.3 |

The locks of hair treated with process 1 according to the invention and washed with five shampoo washes have lower ΔE values than those of the locks of hair treated with comparative processes 2 and 3 respectively.

Thus, the coloured coating obtained with the processes according to the invention shows improved persistence with respect to shampoo washing.

### Example 3

Composition C1 as described below was prepared: the amounts are expressed as g of starting material as obtained/100 g, unless otherwise mentioned. In the table 5 below, “a.m.” means “active material”.

TABLE 5

| Composition | C1 |
|---|---|
| Polycarbodiimide⁽¹⁾ | 24 |
| | 9.6 AM |
| Hydroxyethyl acrylate/sodium acryloyldimethyl | 1 |
| taurate copolymer ⁽²⁾ | 0.9 AM |
| Amodimethicone (and) Trideceth-5 (and) | 5 |
| Trideceth-10 ⁽³⁾ | 0.75 AM |
| Preserving agent(s) | qs |
| Water | qs 100 |

(1) synthesized according to the synthetic process described in Example 1 (containing 40% active material in water),
(2) sold by the company SEPPIC under the name Sepinov EMT10 (containing 90% active material),
(3) sold by the company Wacker under the name Belsil ADM LOG 1 (containing 15% active material).

Compositions D3 and D4 as described below were prepared: the amounts are expressed as g of unmodified starting material/100 g.

TABLE 6

| | D3 | D4 |
|---|---|---|
| Compositions | invention | comparative |
| BIS-CARBOXYDECYL DIMETHICONE | — | 15.12 |
| (silicone elastomer containing carboxylic | | |
| acid ) ⁽⁴⁾ | | |
| Isododecane (and) hexyl/succinyl dimethicone | 50.4 | — |
| crosspolymer⁽⁵⁾ | 15.12 AM | |
| Red mineral pigment | 10 | 10 |
| Isododecane | qs 100 | qs 100 |

(4) sold by the company Momentive Performance Materials under the name Silform INX,
(5) sold by the company Dow Corning under the name Dowsil EL-7314 Silicone Elastomer Blend® (containing 30% active material in isododecane)

### Protocol:

Composition C2 is applied to locks of natural hair containing 90% white hair strands, at a rate of 0.4 g of composition per gram of lock. The locks of hair are then combed and dried with a hairdryer.

Composition D3 or composition D4 is then applied to the locks of hair treated beforehand with composition C1, in a proportion of 0.4 g of composition per gram of lock.

The locks of hair are then combed and dried with a hairdryer.

### Summary of the Various Processes:

| | P1 = | P2 = | P3 = |
|---|---|---|---|
| | Process 1 | Process 2 | Process 3 |
| | Invention | Comparative | Comparative |
| Application of composition C1 | Yes | Yes | No |
| Application of composition D | D1 | D2 | D1 |

The locks of hair thus coloured are then subjected to a test of several repeated shampoo washes so as to evaluate the fastness (persistence) of the colouring obtained with respect to shampoo washes, according to the shampoo washing protocol described below.

### Shampoo Washing Protocol:

The locks of dyed hair are combed, moistened with water at 35° C. and then passed between the fingers five times for 5 seconds. The locks of hair are then squeezed dry between two fingers.

A standard shampoo (Garnier Ultra Doux) is applied uniformly to the coloured locks, in a proportion of 0.4 g of standard shampoo per gram of locks, the locks of hair being massaged gently along the length (6 passes) for 15 seconds, from the root to the end. The locks of hair are then placed on a watch glass and left to stand for 1 minute.

Next, the locks of hair are rinsed with water while passing the locks between the fingers (15 passes). The locks of hair are then squeezed dry between two fingers before the next shampoo wash.

Once the tests of several shampoo washes have been performed, the locks of hair are combed and dried with a hairdryer.

### Results

The persistence of the color of the locks was evaluated in the CIE L*a*b* system, using a Minolta Spectrophotometer CM3600A colorimeter (illuminant D65, angle 10°, specular component included).

In this L*a*b* system, L* represents the intensity of the color, a* indicates the green/red color axis and b* indicates the blue/yellow color axis.

The persistence of the coloring is evaluated by the color difference ΔE between the dyed locks before shampooing, then after having undergone five shampooing operations according to the protocol described above. The lower the ΔE value, the more persistent the colour with respect to shampoo washing.

The ΔE value is calculated according to the following equation:

$$Δ E = \sqrt{{( {L}^{*} - {{L}_{0}}^{*} )}^{2} + {( {a}^{*} - {{a}_{0}}^{*} )}^{2} + {( {b}^{*} - {{b}_{0}}^{*} )}^{2}}$$

In this equation, L*a*b* represent the values measured after colouring the hair and after performing the shampoo washes, and L₀*a₀*b₀* represent the values measured after colouring the hair but before shampoo washing.

TABLE 7

| | Number of | | | | |
|---|---|---|---|---|---|
| | shampoo | | | | |
| Processes | washes | L* | a* | b* | ΔE |
| P1 = C1 + D3 | 0 | 36.8 | 22.8 | 17.3 | — |
| (Invention) | 5 | 53.5 | 11.4 | 14.3 | 20.4 |
| P2 = C1 + D4 | 0 | 35.5 | 30.1 | 25.5 | — |
| (comparative) | 5 | 58.2 | 7.4 | 15.7 | 33.5 |
| P3 = D3 | 0 | 37.4 | 22.0 | 16.9 | — |
| (comparative) | 5 | 58.9 | 3.1 | 13.6 | 28.6 |

The locks of hair treated by means of process 1 according to the invention and washed with five shampoo washes have lower ΔE values than those of the locks of hair treated by means of the comparative processes 2 and 3, respectively.

Thus, the coloured coating that is obtained by means of the processes according to the invention shows improved shampoo-fastness.

### Example 4

Composition C3 as described below was prepared: the amounts are expressed as g of starting material as obtained/100 g, unless otherwise mentioned. In the table 5 below, “a.m.” means “active material”.

TABLE 8

| Composition | C3 |
|---|---|
| Polycarbodiimide⁽¹⁾ | 24 |
| | 9.6 a.m |
| Hydroxyethyl acrylate/sodium | 1 |
| acryloyldimethyl taurate copolymer ⁽²⁾ | 0.9 a.m |
| Amodimethicone (and) Trideceth-5 | 5 |
| (and) Trideceth-10 ⁽³⁾ | 0.75 a.m |
| Preserving agent(s) | qs |
| Water | qs 100 |

TABLE 9

| Composition | C4 |
|---|---|
| Acrylate copolymer latex (30% AM) | 40 |
| Daitosol 3000SLPN-PE1 from Daito Kasei Kogyo | 12 a.m |
| Acrylates/steareth-20 methacrylate copolymer (30% AM) | 2 |
| Aculyn 22, Rohm & Haas | 0.6 a.m |
| Triethanolamine | 0.67 |
| Amodimethicone (and) Trideceth-5 (and) Trideceth-10 ⁽³⁾ | 5 |
| | 0.75 a.m |
| Red mineral pigment | 12 |
| Preserving agent(s) | qs |
| Water | qs 100 |

(3) sold by the company Wacker under the name Belsil ADM LOG 1 (containing 15% active material).

TABLE 10

| Compositions | D5 | D6 |
|---|---|---|
| | invention | comparative |
| BIS-CARBOXYDECYL DIMETHICONE | — | 2 |
| (silicone elastomer containing carboxylic | | |
| acid) ⁽⁴⁾ | | |
| Isododecane (and) hexyl/succinyl dimethicone | 6.66 SM | — |
| crosspolymer⁽⁵⁾ | 2 a.m | |
| Isododecane | qs 100 | qs 100 |

(4) sold by the company Momentive Performance Materials under the name Silform INX,
(5) sold by the company Dow Corning under the name Dowsil EL-7314 Silicone Elastomer Blend® (containing 30% active material in isododecane)

### Protocol:

Composition C′ is prepared by mixing composition C3 and composition C4 in a 1+1 ratio before application.

Composition C′ is then applied to locks of natural hair containing 90% white hair strands, at a rate of 0.8 g of composition per gram of lock. The locks of hair are then combed and dried with a hairdryer.

Composition D5 or composition D6 is then applied to the locks of hair treated beforehand with composition C′, in a proportion of 0.5 g of composition per gram of lock.

The locks of hair are then combed and dried with a hairdryer.

The locks of hair thus dyed are then subjected to a test of several repeated shampoo washes so as to evaluate the fastness (persistence) of the colouring obtained with respect to shampoo washes, according to the shampoo washing protocol described above.

### Results

TABLE 11

| | Number of | | | | |
|---|---|---|---|---|---|
| | shampoo | | | | |
| Processes | washes | L* | a* | b* | ΔE |
| P1 = C′ + D5 | 0 | 35.8 | 25.7 | 19.5 | — |
| (Invention) | 5 | 37.0 | 23.7 | 18.0 | 2.8 |
| P2 = C′ + D6 | 0 | 34.9 | 25.9 | 20.2 | — |
| (comparative) | 5 | 39.6 | 22.0 | 16.6 | 7.1 |

The locks of hair treated by means of process 1 according to the invention and washed with five shampoo washes have lower ΔE values than those of the locks of hair treated by means of comparative process 2.

Thus, the coloured coating that is obtained by means of the processes according to the invention shows improved shampoo-fastness.

1. Process for colouring the hair comprising the following steps:
a) the application to the hair of at least one composition C comprising:
one or more (poly)carbodiimide compounds; and
b) the application to the hair of at least one composition D comprising at least one silicone elastomer containing carboxylic acid functions;
composition C and/or composition D comprising at least one colouring agent chosen from pigments, direct dyes and mixtures thereof.

2. Process according to claim 1, characterized in that the (poly)carbodiimide compound(s) are chosen from the compounds of formula (I) below: 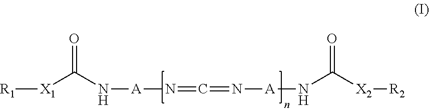
in which:
X₁ and X₂ independently represent an oxygen atom O, a sulfur atom S or an NH group;
R₁ and R₂ independently represent a group chosen from a hydrocarbon-based radical, preferably alkyl, optionally interrupted with one or more heteroatoms, a group chosen from alkoxysilyl, hydroxysilyl, acetoxysilyl, vinylsilyl, acrylalkylsilyl, methacrylalkylsilyl, crotonylalkylsilyl, carboxyanhydridoalkylsilyl, carboxyalkylsilyl, hydroxyalkylsilyl, aldehydoalkylsilyl, mercaptoalkylsilyl, norbornenylsilyl, acylpentadienylalkylsilyl, maleimidoalkylsilyl, sulfonylalkylsilyl, (meth)acrylalkyl, crotonylalkyl, alkylepoxide such as propylepoxide or butylepoxide and azacyclopropane groups, and a hydrocarbon-based radical, preferably alkyl, optionally interrupted with one or more heteroatoms and with one or more groups chosen from alkoxysilyl, hydroxysilyl, acetoxysilyl, vinylsilyl, acrylalkylsilyl, methacrylalkylsilyl, crotonylalkylsilyl, carboxyanhydridoalkylsilyl, carboxyalkylsilyl, hydroxyalkylsilyl, aldehydoalkylsilyl, mercaptoalkylsilyl, norbornenylsilyl, acylpentadienylalkylsilyl, maleimidoalkylsilyl, sulfonylalkylsilyl, (meth)acrylalkyl, crotonylalkyl, alkylepoxide such as propylepoxide or butylepoxide and azacyclopropane groups;
n denotes an integer ranging from 1 to 1000; and
A is a monomer chosen from the compounds below:

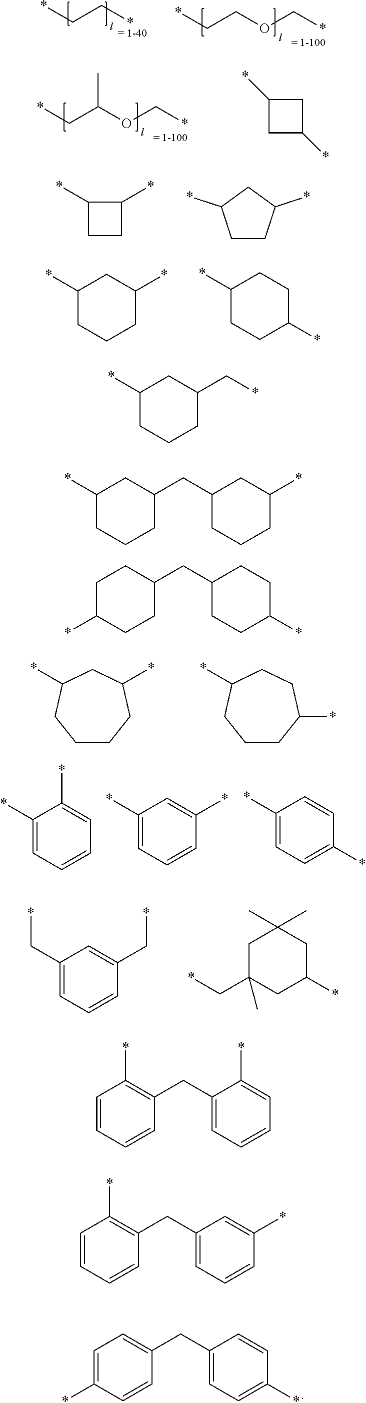

3. Process according to claim 1, characterized in that the (poly)carbodiimide compound(s) are chosen from the compounds of formula (II) below: 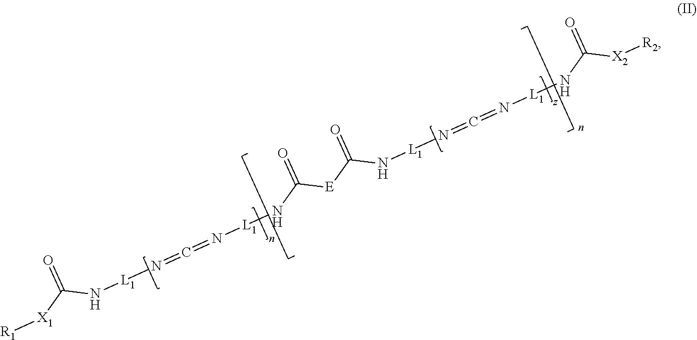
in which:
X₁ and X₂ independently represent an oxygen atom O, a sulfur atom S or an NH group;
R₁ and R₂ independently represent a hydrocarbon-based radical optionally interrupted with one or more heteroatoms;
n and z denote an integer ranging from 1 to 20, with n+z≥2 and w denotes an integer ranging from 1 to 3;
L₁ independently represents a C₁-C₁₈ divalent aliphatic hydrocarbon-based radical, a C₃-C₁₅ cycloalkylene radical, a C₃-C₁₂ heterocycloalkylene group or a C₆-C₁₄ arylene group, and mixtures thereof;
E independently represents a group chosen from:
—O—R₃—O—; —S—R₄—S—; —R₅—N(R₆)—R₄—N(R₆)—R₅—;
in which R₃ and R₄ independently represent a divalent hydrocarbon-based radical optionally interrupted with one or more heteroatoms;
R₅ independently represents a covalent bond or a saturated divalent hydrocarbon-based radical, optionally interrupted with one or more heteroatoms;
R₆ independently represents a hydrogen atom or a hydrocarbon-based radical, optionally interrupted with one or more heteroatoms.

4. Process according to claim 3, characterized in that the (poly)carbodiimide compound(s) are chosen from the compounds of formula (II) in which:
X₁ and X₂ independently represent an oxygen atom;
R₁ and R₂ are independently chosen from dialkylamino alcohols, alkyl esters of hydroxycarboxylic acid and monoalkyl ethers of (poly)alkylene glycol, in which a hydroxyl group has been removed, and mixtures thereof;
n and z denote an integer ranging from 1 to 20, with n+z≥2 and w is equal to 1;
L₁ is chosen from a C₁-C₁₈ divalent aliphatic hydrocarbon-based radical, a C₃-C₁₅ cycloalkylene radical, a C₃-C₁₂ heterocycloalkylene group or a C₆-C₁₄ arylene group, and mixtures thereof;
E independently represents a group chosen from:
—O—R₃—O—; —S—R₄—S—; —R₅—N(R₆)—R₄—N(R₆)—R₅—;
in which R₃ and R₄ are independently chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof;
when R₅ is not a covalent bond, R₅ is chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof; and
R₆ is chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof.

5. Process according to claim 3, characterized in that the (poly)carbodiimide compound(s) are chosen from the compounds of formula (II) in which:
X₁ and X₂ independently represent an oxygen atom;
R₁ and R₂ are, independently, monoalkyl ethers of (poly)alkylene glycol, in which a hydroxyl group has been removed;
n and z denote an integer ranging from 1 to 20, with n+z≥2 and w is equal to 1;
L₁ is a C₃-C₁₅ cycloalkylene radical;
E independently represents a group chosen from:
—O—R₃—O—; —S—R₄—S—; —R₅—N(R₆)—R₄—N(R₆)—R₅—;
in which R₃ and R₄ are independently chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof;
when R₅ is not a covalent bond, R₅ is chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof; and
R₆ is chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof.

6. Process according to claim 3, characterized in that the (poly)carbodiimide compound(s) are chosen from the compounds of formula (II) in which:
X₁ and X₂ independently represent an oxygen atom;
R₁ and R₂ independently represent the compound of formula (VI) below:
R₁₃—[O—CH₂—C(H)(R₁₄)]_{q}— (VI),
in which R₁₃ represents a C₁-C₄ alkyl group or a phenyl, preferably a C₁-C₄ alkyl group, more preferentially a methyl, R₁₄ represents a hydrogen atom or a C₁-C₄ alkyl group, preferably a hydrogen atom and q denotes an integer ranging from 4 to 30;
n and z denote an integer ranging from 2 to 20, with n+z ranging from 4 to 10 and w is equal to 1;
L₁ is a C₃-C₁₅ cycloalkylene radical such as cyclopentylene, cycloheptylene, cyclohexylene and 4,4-dicyclohexylenemethane; and
E represents a group —O—R₃—O— in which R₃ is chosen from a C₆-C₁₄ arylene radical, a C₃-C₁₂ cycloalkylene radical, a linear or branched C₁-C₁₈ alkylene radical, optionally interrupted with one or more heteroatoms, and mixtures thereof.

7. Process according to claim 3, characterized in that the (poly)carbodiimide compound(s) are chosen from the compounds of formula (II) in which:
X₁ and X₂ independently represent an oxygen atom;
R₁ and R₂ independently represent the compound of formula (VI) below:
R₁₃—[O—CH₂—C(H)(R₁₄)]_{q}— (VI),
in which R₁₃ represents a C₁-C₄ alkyl group or a phenyl, preferably a C₁-C₄ alkyl group, more preferentially a methyl, R₁₄ represents a hydrogen atom or a C₁-C₄ alkyl group, preferably a hydrogen atom and q denotes an integer ranging from 4 to 30;
n and z denote an integer ranging from 2 to 20, with n+z ranging from 4 to 10 and w is equal to 1;
L₁ is a C₃-C₁₅ cycloalkylene radical such as cyclopentylene, cycloheptylene, cyclohexylene and 4,4-dicyclohexylenemethane, preferably 4,4-dicyclohexylenemethane; and
E represents a group —O—R₃—O— in which R₃ represents a linear or branched C₁-C₁₈ alkylene radical such as methylene, propylene, butylene or ethylene, optionally interrupted with one or more heteroatoms.

8. Process according to claim 3, characterized in that the (poly)carbodiimide compound(s) are chosen from the compounds of formula (XII) below: 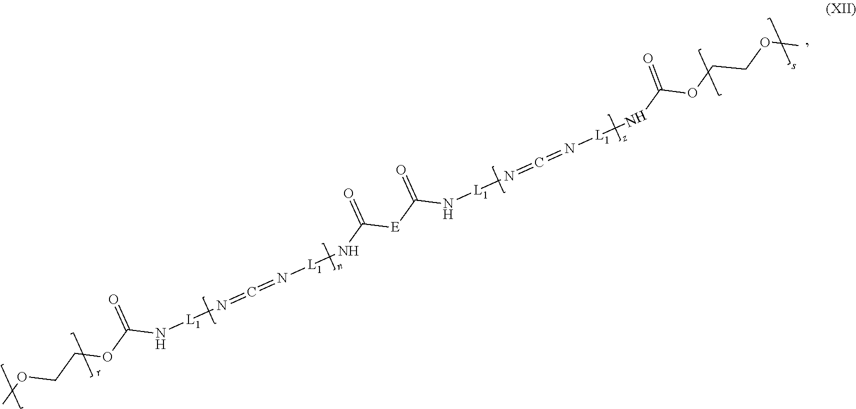
in which L₁ is 4,4-dicyclohexylenemethane, n and z denote an integer ranging from 2 to 20, with n+z ranging from 4 to 10, E represents a group —O—R₃—O— in which R₃ represents a linear or branched C₁-C₁₈ alkylene radical such as methylene, propylene, butylene or ethylene, optionally interrupted with one or more heteroatoms, and r and s denote an integer ranging from 4 to 30.

9. Process according to claim 1, characterized in that the total amount of the (poly)carbodiimide compound(s) ranges from 0.01% to 40% by weight, preferably from 0.5% to 30% by weight, more preferentially from 1% to 20% by weight, even more preferentially from 2% to 15% by weight and better still from 3% to 10% by weight, relative to the total weight of composition C.

10. Process according to claim 1, characterized in that the silicone elastomer bearing carboxylic acid functions is chosen from those corresponding to formula (XIII) below: 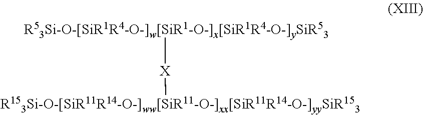
in which:
the radical X corresponds to the formula (i) below: 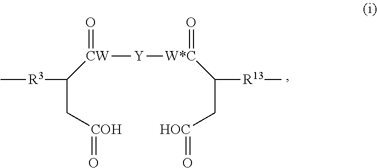
or to formula (i′) below: 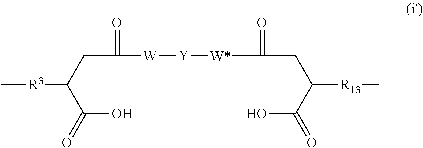
in which:
W and W* independently denote an oxygen atom (O) or a group N—R in which each radical R independently denotes a hydrogen atom (H) or a radical R¹;
Y is a divalent group;
R¹, R¹¹, R⁴, R¹⁴, R⁵ and R¹⁵ independently denote a linear or branched, saturated or unsaturated, cyclic or acyclic, substituted or unsubstituted hydrocarbon-based radical;
R³ and R¹³ independently denote a divalent group;
w and ww are independently integers ranging from 0 to 1000.
x and xx are independently integers ranging from 1 to 100;
y and yy are independently integers ranging from 0 to 1000;
preferably, w and y are not simultaneously equal to 0 and ww and yy are not simultaneously equal to 0.

11. Process according to claim 10, characterized in that the silicone elastomer of formula (XIII) is chosen from compounds 1 to 12 below:

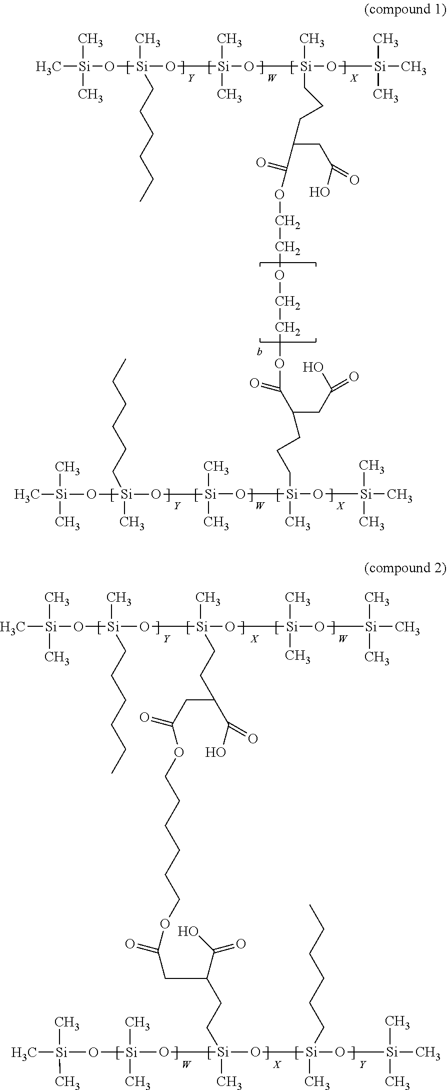

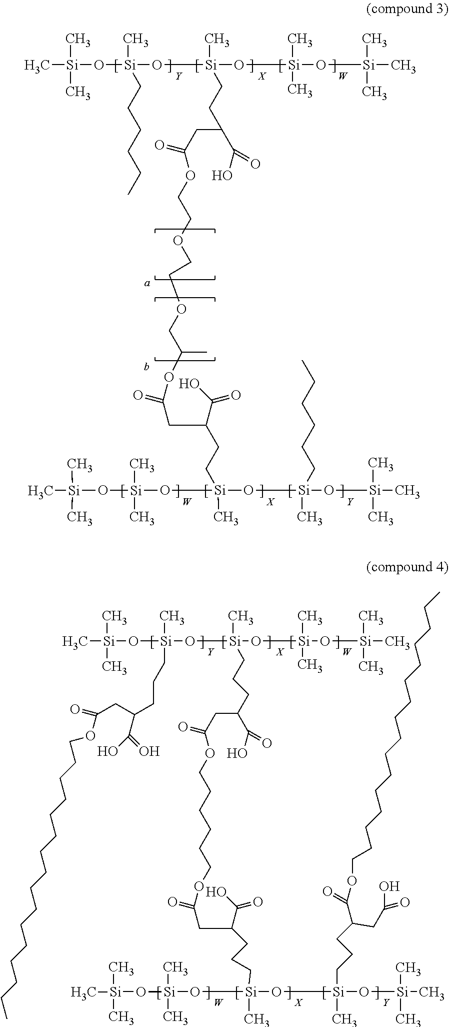

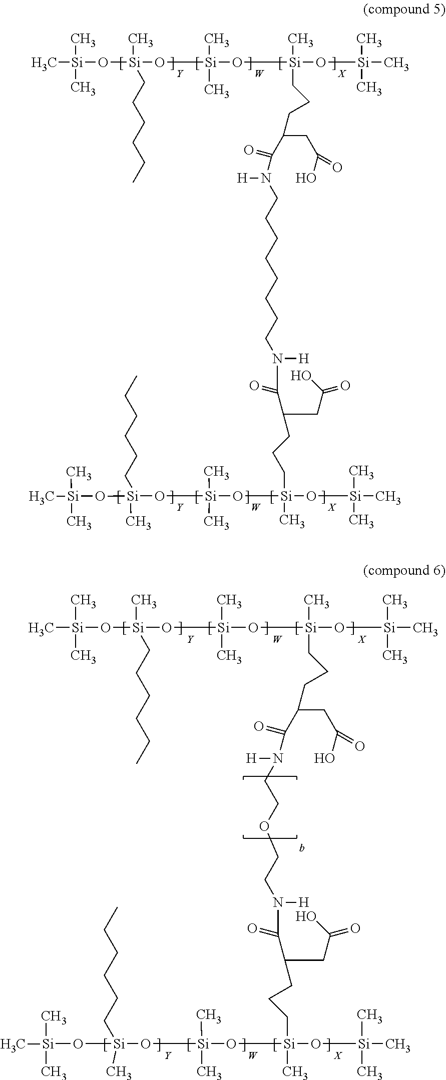

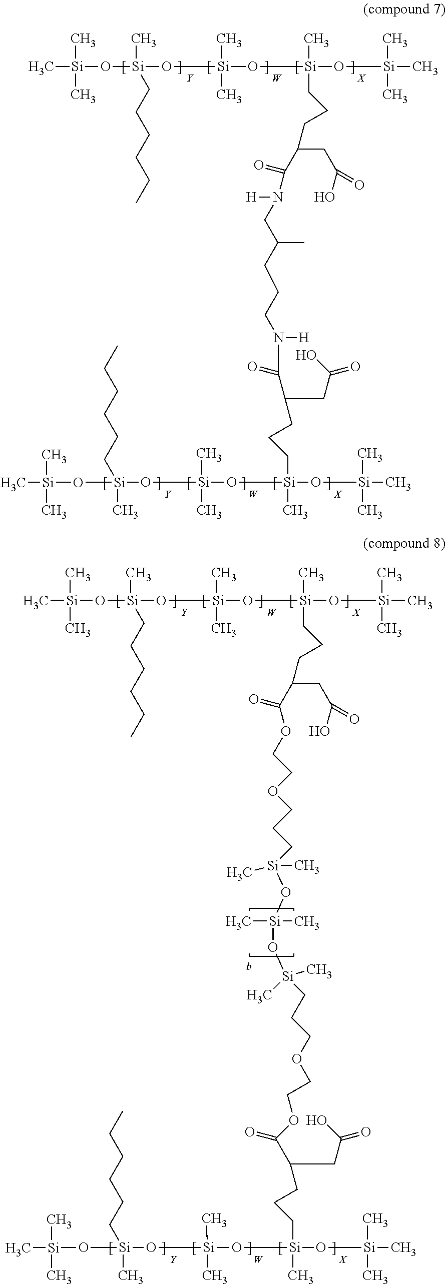

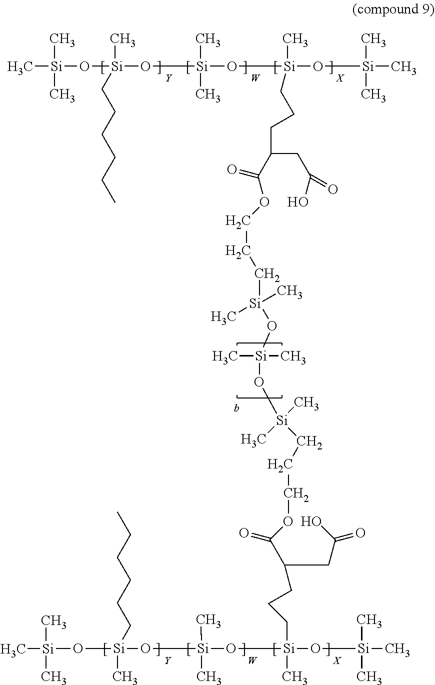

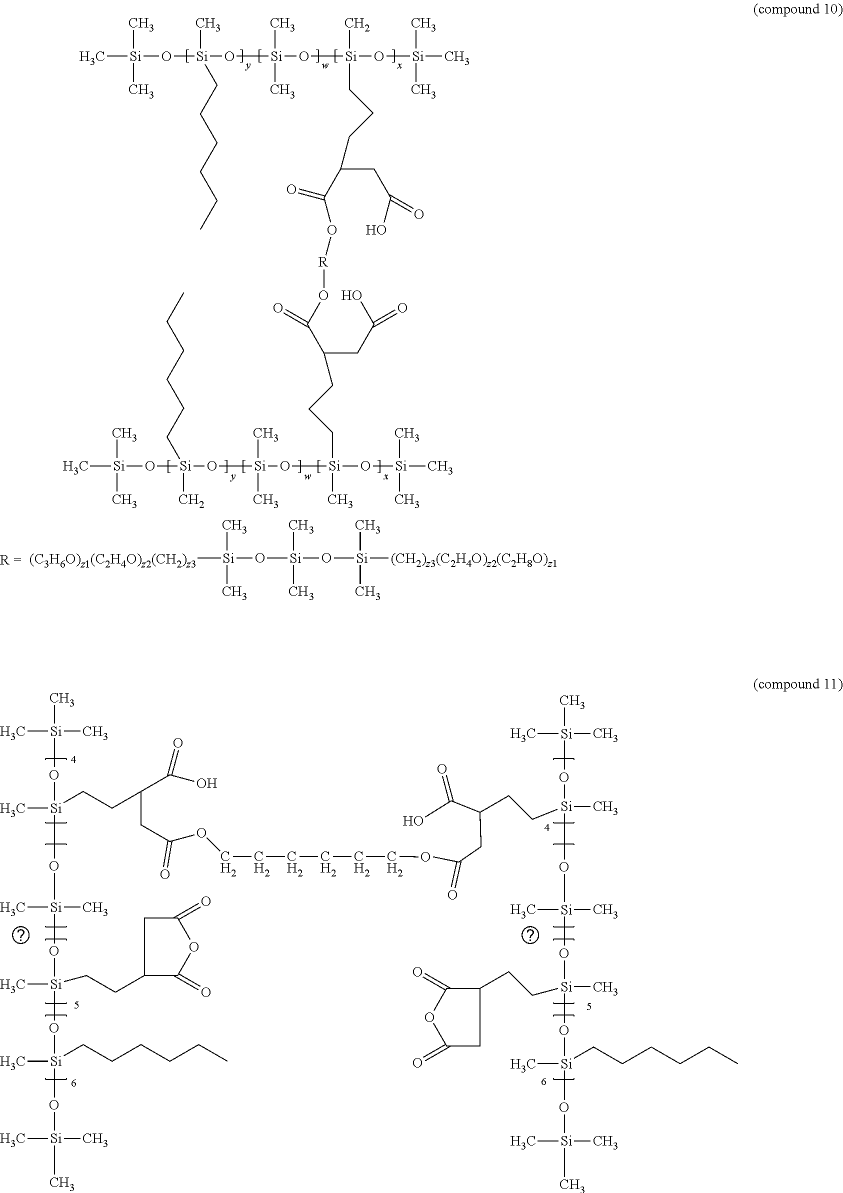

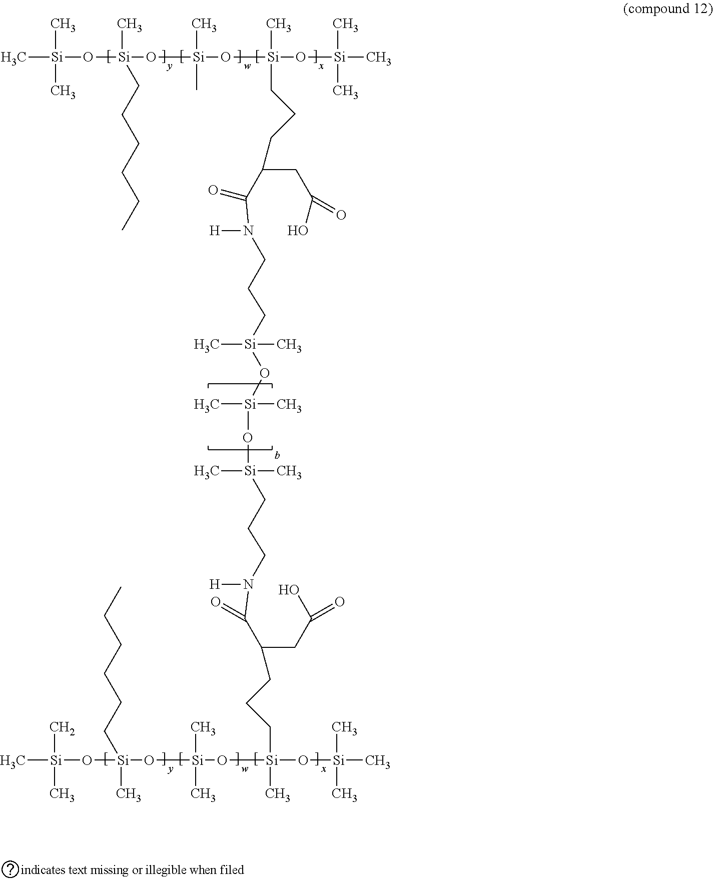

12. Process according to claim 11, characterized in that the silicone elastomer bearing carboxylic acid functions is compound 11.

13. Process according to claim 1, characterized in that the silicone elastomer bearing carboxylic acid functions is in dispersion in at least one oil and in gel form; preferably chosen from volatile hydrocarbon-based oils, non-volatile hydrocarbon-based oils, and mixtures thereof, preferably isododecane.

14. Process according to claim 13, characterized in that the oily dispersion of silicone elastomer bearing carboxylic acid functions comprises from 10% to 40% by weight of silicone elastomer active material, and more preferably from 20% to 35% by weight of silicone elastomer active material, relative to the total weight of the oily dispersion.

15. Process according to claim 1, characterized in that the silicone elastomer containing carboxylic acid functions is present in a content ranging from 0.1% to 40% by weight, preferably from 1% to 35% by weight, more preferentially from 5% to 30% by weight and better still from 10% to 25% by weight, relative to the weight of composition D.

16. Device for colouring the hair comprising at least two compartments containing:
in a first compartment, a composition C comprising:
one or more (poly)carbodiimide compounds as defined in claim 2; and
in a second compartment, a composition D comprising at least one silicone elastomer containing carboxylic acid functions;
composition C and/or composition D comprising at least one colouring agent chosen from pigments, direct dyes and mixtures thereof.
